(19) United States
(12) Patent Application Publication
(10) Pub. No.: US 2023/0334552 A1
(43) Pub. Date: Oct. 19, 2023

(54) SYSTEMS AND METHODS FOR RECOMMENDING INGREDIENTS AND PRODUCTS

(71) Applicant: Jacqueline M. LEVIN, Laguna Nigel, CA (US)

(72) Inventor: Jacqueline M. LEVIN, Laguna Nigel, CA (US)

(21) Appl. No.: 18/247,828

(22) PCT Filed: Jan. 12, 2022
(86) PCT No.: PCT/US2022/012101

§ 371 Date: Apr. 4, 2023

Related U.S. Application Data
(60) Provisional application No. 63/199,628, filed on Jan. 13, 2021.

(51) Int. Cl. G06Q 30/0601; G16H 20/10; G16H 50/30; A45D 44/00

(52) U.S. Cl. CPC G06Q 30/0631; G16H 20/10; G16H 50/30; A45D 44/005; G16H 10/60

(57) ABSTRACT

The present disclosure provides systems and methods for recommending ingredients and/or products. In an aspect, the present disclosure provides a method for recommending ingredients and/or products. The method may comprise (a) receiving information about a user, wherein the information comprises (i) genetic data of the user, (ii) user responses to a health and profile survey, and (iii) user inputs corresponding to one or more ingredients to avoid; (b) using a user analysis algorithm to generate one or more user attributes based on the information about the user; (c) correlating the one or more user attributes to one or more ingredient effects associated with one or more reference ingredients; and (d) using the correlations between the one or more user attributes and the one or more ingredient effects to generate (i) a preliminary ingredient avoid list.

### CROSS REFERENCE

This application claims priority to U.S. Provisional Patent Application No. 63/199,628 filed on Jan. 13, 2021, which application is incorporated herein by reference in its entirety for all purposes.

### BACKGROUND

Skincare technology is a booming business, with present worldwide revenues in excess of $130 billion. The skincare industry is traditionally characterized by large companies that file a lot of patent applications to protect their intellectual property. Just seven companies produce nearly two hundred of the most recognized beauty brands in the world; L′Oréal, Johnson and Johnson, Shiseido, Estee Lauder Companies, Unilever, Coty, and Procter & Gamble. Skincare technology is one of the leading categories of new patent filings. Recently, there has been a proliferation of patent filings for advancements in “clean beauty” technology as consumers have responded favorably to the availability of skincare products formulated with ingredients that are intended to cause no harm.

Although commercial applications for identifying and recommending clean beauty products have proliferated, “clean beauty” itself is a misnomer. There is no universal definition of “clean beauty,” it is in the eye of the beholder in the context of his or her own personal experience. In addition, “clean beauty” only addresses a few select medical conditions that are supposed to be universal and/or relevant to a large subset of the human population. Yet for many, those conditions are not relevant and the health conditions/goals that are relevant to a particular user may not be addressed in clean beauty recommendations that are available today. Therefore, modern concepts of clean beauty do not address the whole person or take into account all medical conditions relevant to a user or a consumer. Furthermore, clean beauty recommendations and concerns today are generally limited to cosmetics and personal care products, when really any topical product including prescriptions and the like should be included. Any topical product can be a viable clean beauty solution to one person and yet cause problems and/or concerns to another person. Such problems and/or concerns may include, for example, health problems or safety concerns. In some cases, such problems and/or concerns may be related to the fact that a topical product may hinder, contradict, or conflict with a person's consumer goals or health goals (e.g., cosmetic effects and/or improvements sought). Unfortunately, trial and error has been the only way for a consumer to determine the efficacy of a touted clean beauty product for herself.

Improvements are needed that would enable the user to avoid purchasing any harmful skincare product on her own personal basis. Such improvements would provide everyone the ability to individually define for themselves what are and what are not clean beauty products. Such improvements would finally lend a universally understandable meaning of the term “clean beauty products.” It is to those improvements that embodiments of the present technology are directed.

### SUMMARY

The present application relates generally to the field of skincare, make up, haircare, nail care, personal care, and topical prescription products, and more particularly without limitation, to e-commerce technology providing the user with a unique, individually tunable, electronic store from which to make informed purchasing decisions of such products.

Recognized herein are various limitations with beauty product recommendation platforms currently available. Current e-commerce platforms attempt to address consumer needs by giving consumers generalized advice about what to avoid and what to use. Such commercially available platforms do not offer user analysis and do not consider the individual user's personal characteristics, such as their age, skin and health history, DNA, goals, and/or health concerns, before offering ingredient and product recommendations. Such platforms also do not provide personalized product recommendations based on a user's age, health risk, allergies, goals and concerns and do not offer a simple “yes” or “no” answer to the question ‘is a product right and/or safe for me in view of any problems and/or concerns I may have?’, which may be confusing to users. Commercially available platforms also fail to consider all of a user's health conditions, and may only consider a select few which may not provide a holistic picture of the user's health. Further, major retailers do not provide consumers with custom ingredient and product recommendations beyond the user's superficial goals. Such major retailers may, in some limited cases, help users avoid specific ingredients they know they want to avoid, but only if the user inputs a specific ingredient concern or knows what ingredients they want to avoid. Such platforms may only provide a very limited selection of ingredients and/or cosmetic products, and do not provide recommendations for ingredients and/or cosmetic products to use and/or avoid based on any analysis of a user's attributes.

The systems and methods of the present disclosure addresses at least the abovementioned shortcomings of conventional beauty product recommendation platforms by providing consumers with personalized ingredient avoid lists and product recommendations based on an in-depth analysis of one or more attributes associated with each consumer. The systems and methods of the present disclosure may provide such personalized ingredient avoid lists and product recommendations based on a consideration of an individual user's medical history, skin type, DNA, allergies, age, and/or risk factors.

In one aspect, the present disclosure provides a method for generating recommendations for ingredients and/or products (e.g., cosmetic products or topical products such as cosmetic products, prescription products, OTC products, make up, hair care, nail care, and personal care products). The method may comprise (a) receiving information about a user, wherein the information comprises (i) genetic data of the user, (ii) user responses to a health and profile survey, and (iii) user inputs corresponding to one or more ingredients to avoid; (b) using a user analysis algorithm to generate one or more user attributes based on the information about the user; (c) correlating the one or more user attributes to one or more ingredient effects associated with one or more reference ingredients; and (d) using the correlations between the one or more user attributes and the one or more ingredient effects to generate (i) a preliminary ingredient avoid list. In some embodiments, the health/profile survey may be used to gather information on a user's age, ethnicity, lifestyle, skin/hair/nail concerns, skin/hair/nail goals, skin/hair/nail type, complete health history, reproductive history and goals, allergies (e.g., environmental, food, drug, and/or skin allergies), genetic concerns, individual risk factors, and wellness and/or product ingredient concerns. In addition, when relevant, the health/profile survey may be used to gather information on location/relevance of the user's goals and concerns (e.g., is acne on the face or back of the user).

In some embodiments, the method may further comprise generating a suggested ingredient avoid list by adding one or more cross reactors to the preliminary ingredient avoid list, wherein the one or more cross reactors comprise ingredients with a chemical structure similar to that of one or more ingredients in the preliminary ingredient avoid list. Identifying cross reactors (i.e., ingredients that are chemically similar to other ingredients that may cause or exacerbate health conditions in a user) can help to warn, inform, or alert users about potential allergic reactions that the user may experience due to the presence of certain ingredients in products.

In some embodiments, the method may further comprise generating a final ingredient avoid list by modifying the suggested ingredient avoid list based on one or more manual adjustments performed by the user. In some embodiments, the method may further comprise identifying special condition avoid list ingredients. For instance, one or more ingredients on the avoid list may be conditional and may only require avoidance when certain product attributes/types and locations of use apply. In one example, an ingredient that worsens glaucoma only needs to be avoided in a product used around the eye. In another example, a respiratory irritant may only be relevant in a spray product.

In some embodiments, the suggested and/or final ingredient avoid list may be generated based on the correlations between the one or more user attributes and the one or more ingredient effects. For example, the analysis may not only consider individual answers but the grouping or combination of answers to determine user attributes. In some cases, an additional layer of analysis may take into account what is communicated in the survey and the assigned user attributes. For each survey answer, the analysis may involve interpreting the location of relevance, aspects of aging/disease pathogenesis, signs and symptoms of aging/disease states, and/or risks factors associated with aging/disease states, and relating these factors to evidence based mechanisms and effects of ingredients, in order to determine which ingredients to include or exclude on the avoid list.

In some embodiments, the method may further comprise generating a preliminary suggested ingredient list based on the correlations between the one or more user attributes and the one or more ingredient effects, wherein the preliminary suggested ingredient list comprises one or more ingredients with therapeutic effects.

In some embodiments, the method may further comprise generating an updated suggested ingredient list based on one or more user inputs corresponding to the user's favorite or preferred ingredients.

In some embodiments, the method may further comprise generating a final suggested ingredient list by subtracting the final ingredient avoid list from the user updated suggested ingredient list.

In some embodiments, the method may further comprise comparing (i) a list of ingredients associated with one or more products against (ii) the final ingredient avoid list and the final suggested ingredient list to generate (iii) one or more product recommendations. In some embodiments, the method may comprise analyzing and/or looking for hazardous combinations of ingredients, ingredient compatibility, or ingredient risks in individual products and groupings of products (sets or regimens) to generate or update product recommendations. In some embodiments, the method may comprise generating the one or more product recommendations based at least in part on the intended area for product use or other product attributes. In some embodiments, the method may comprise generating the one or more product recommendations based at least in part on whether certain conditions relating to special condition avoid list ingredients are satisfied or likely to be satisfied. In some cases, if certain conditions are satisfied or likely to be satisfied, one or more special condition avoid list ingredients may be included or excluded from the one or more product recommendations.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates a generalized block diagram of skincare management technology, in accordance with some embodiments.

FIG. 2 schematically illustrates a device configured to practice skincare or any topical product management technology, in accordance with some embodiments.

FIG. 3 schematically illustrates a flowchart depicting steps in a method for practicing the user filter step in FIG. 1, in accordance with some embodiments.

FIG. 4 schematically illustrates the user's attribute profile, in accordance with some embodiments.

FIG. 5 schematically illustrates indexing the attribute database in relation to the user's attribute profile, in accordance with some embodiments.

FIG. 6 schematically illustrates obtaining the set of unfavorably correlated ingredients from the indexing operation of FIG. 5, in accordance with some embodiments.

FIG. 7 schematically illustrates a flowchart depicting steps in a method for practicing the product filter step in FIG. 1, in accordance with some embodiments.

FIG. 8 schematically illustrates a flow chart corresponding to a method for generating personalized ingredient lists and personalized product recommendations, in accordance with some embodiments.

FIG. 9 schematically illustrates a user analysis algorithm, in accordance with some embodiments.

FIG. 10 schematically illustrates a product analysis algorithm, in accordance with some embodiments.

FIGS. 11-28 schematically illustrate a user interface for implementing the systems and methods of the present disclosure, in accordance with some embodiments.

FIG. 29 schematically illustrates a computer system that is programmed or otherwise configured to implement methods provided herein.

FIG. 30 schematically illustrates an exemplary user interface for viewing and browsing products, in accordance with some embodiments.

FIGS. 31-32 schematically illustrate an exemplary user interface for viewing product and ingredient information, in accordance with some embodiments.

FIGS. 33-34 schematically illustrate an exemplary user interface for viewing ingredients in an ingredient library and associated ingredient information, in accordance with some embodiments.

FIGS. 35-36 schematically illustrate an exemplary user interface for viewing and managing a user's personalized regimen, in accordance with some embodiments.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “real time” or “real-time,” as used interchangeably herein, generally refers to an event (e.g., an operation, a process, a method, a technique, a computation, a calculation, an analysis, a visualization, an optimization, etc.) that is performed using recently obtained (e.g., collected or received) data. In some cases, a real time event may be performed almost immediately or within a short enough time span, such as within at least 0.0001 millisecond (ms), 0.0005 ms, 0.001 ms, 0.005 ms, 0.01 ms, 0.05 ms, 0.1 ms, 0.5 ms, 1 ms, 5 ms, 0.01 seconds, 0.05 seconds, 0.1 seconds, 0.5 seconds, 1 second, or more. In some cases, a real time event may be performed almost immediately or within a short enough time span, such as within at most 1 second, 0.5 seconds, 0.1 seconds, 0.05 seconds, 0.01 seconds, 5 ms, 1 ms, 0.5 ms, 0.1 ms, 0.05 ms, 0.01 ms, 0.005 ms, 0.001 ms, 0.0005 ms, 0.0001 ms, or less.

In an aspect, the present disclosure provides a method for generating recommendations for ingredients or products (e.g., cosmetic products or topical products such as cosmetic products, prescription products, OTC products, and personal care products). The method may comprise (a) receiving information about a user, wherein the information comprises (i) genetic data of the user, (ii) user responses to a health and profile survey, and (iii) user inputs corresponding to one or more ingredients to avoid, wellness concerns, allergies, and/or lifestyles; (b) using a user analysis algorithm to generate one or more user attributes based on the information about the user; (c) correlating the one or more user attributes to one or more ingredient effects (some of which may be location dependent) associated with one or more reference ingredients; and (d) using the correlations between the one or more user attributes and the one or more ingredient effects to generate (i) a preliminary ingredient avoid list. In some embodiments, the health/profile survey may be used to gather information on a user's age, ethnicity, lifestyle, skin/hair/nail concerns, skin/hair/nail goals, skin/hair/nail type, complete health history, reproductive history and goals, allergies (e.g., environmental, food, drug, and/or skin allergies), genetic concerns, individual risk factors, and wellness and/or product ingredient concerns. In addition, when relevant, the health/profile survey may be used to gather information on location/relevance of the user's goals and concerns (e.g., is acne on the face or back of the user).

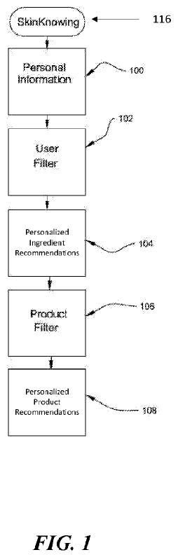

FIG. 1 schematically illustrates a high level generalization of the present technology, generally directed to various non-limiting aspects and embodiments of skincare systems and associated methodology that revolutionizes the way people choose or select their skin care, make up, topical prescription, hair care, nail care, and/or personal care products. This technology utilizes local and remote computer processing power to arm the user with consistently accurate, and individually tunable, knowledge of which skincare products and corresponding ingredients are likely to personally cause harm or provide certain potentially therapeutic benefits.

Consumers have become more conscious of health and environmental ramifications in play when it comes to purchasing their beauty, skin care, topical prescription, and/or personal care products. Generally, consumers are demanding to know “what products are safe to use?” or “what ingredients should I avoid?” Unfortunately, there is no solution in the industry answering these questions on a personal, individual basis. Instead, so-called “clean beauty” technology only provides generalized cookie-cutter advice on what to avoid and what to use. For example, the “No List” (Honest Company) and the “Never List” (Beauty Counter). However these cookie cutter avoid lists only address a few health and wellness concerns and in reality, recommending what ingredients a person should avoid is impossible to generalize for every person and their unique skin goals, health and genetic risks, wellness concerns, and allergies, since the whole person and all of their relevant health concerns, risk and goals should be considered.

As for the popular clean beauty movement, all the emphasis tends to be on what beneficial ingredient has been added to a skincare product, or perhaps what harmful ingredient has been removed. There is too little emphasis on what else is in that skincare product. The product label itself, in the list of ingredients, is pretty much it. The general unavailability of clear and consistent ingredient information frustrates many commercial transactions, especially among sophisticated online consumers who′d rather not resort to physically reading product labels at a retail store in order to make a purchase.

Some attempts have been made to help the consumer avoid skincare products containing certain specific ingredients. For example, commercially available systems like Hello Avo and Naked Poppy may offer limited personalized advice on which products and sometimes ingredients to use; however, their recommendations or analysis is typically no more extensive than, for instance, a retailor recommending wrinkle products in response to a user's stated goal of treating her wrinkles. However, no solution is available that provides the consumer with a personalized “OK to use” list based on results of a user analysis that takes into consideration individual factors such as the user's complete medical history, skin type, DNA, allergies, age, wellness concerns, and other risk factors. Further, no solution is available that answers the user's question “is it safe?” straightforwardly with a simple “yes” or “no” answer.

The present disclosure provides systems and methods that enable consumers to achieve their own Clean Beauty goals on a personalized and individual level. The systems and methods may be implemented to provide ingredient and/or product recommendations that are personalized to each individual consumer's goals and user attributes. Such user attributes may comprise (i) a set of attributes derived from one or more survey input and (ii) a set of interpreted or inferred user attributes derived from the set of attributes initially determined from a user's survey inputs. The interpreted or inferred user attributes may comprise attributes that can be derived or inferred based on a correlation or interaction between two or more existing user attributes. The combination of (i) and (ii) may determine a final set of user attributes that can be taken into account before providing an ingredient and/or product recommendation.

Returning to FIG. 1, the present skincare technology first gathers personal information about the user in block 100. In illustrative embodiments, the user can complete a health and profile survey soliciting information about such things as the user's age, ethnicity, sun exposure, lifestyle risks & habits, skin/hair/nail type, skin/hair/nail concerns/goals, and health history, allergies (food, drug, environment, skin and/or specific ingredients and or ingredient families), genetics or genetic concerns, individual risk factors, family history, reproductive history and goals, wellness and/or product goals (including, for example, skin, hair, and/or nail goals), wellness and/or product ingredient concerns, and the like. In addition, when relevant, the systems and methods may be configured or implemented to gather information on the location/relevance of the user's goals and concerns (e.g., location of acne on the face or the back of the user) and in some cases a measure of their level of concern. In other cases, the user information for the profile and health survey can be sourced partially or completely from other software systems, electronic health/medical records, and/or one or more application programming interface (APIs). This personal information can be inputted into a user filter computer application 102 which, in turn, interprets/analyzes the health and profile answers and creates one or more personalized ingredient recommendations 104 for the user's own unique, individual circumstances. In some embodiments, the user filter computer application 102 determines user attributes based on survey answers (actual check boxes tell us health history, genetic, allergies, lifestyles, goals and concerns) and interprets or analyzes those answers (e.g., their genetic and health risks, symptoms and pathogenesis and associations of the their goals, concerns and health problems, mechanisms that will exacerbate or ameliorate their skin hair nail goals and these risks) to further determine user attributes beyond what is communicated by the user. For example, if a person has eczema on the arms, the user filter computer application 102 can interpret that the person has risks for dry skin, irritated skin, increased risk for certain allergies, inflammation skin, itchy skin, etc. and interpret or infer pathways of the skin that are overexpressed, or malfunctions such as decreased filaggrin and ceramides, altered skin pH, impaired barrier function and repair, altered buffered capacity, and increased inflammatory pathways. In another example, the user filter computer application 102 can also interpret combinations of answers to determine a user's attributes. For instance, if a user just has high blood pressure versus a person who has high blood pressure plus high cholesterol, history of angina or heart attack risks would be determined differently and therefore recommendations would be interpreted differently. In some embodiments, the user filter creates both a personalized list of ingredients that are recommended as OK to use, a personalized list of ingredients to use for their therapeutic value, and a personalized list of ingredients that should be avoided. Avoid list ingredients represent ingredients that will increase that user's risk, are counterintuitive or counterproductive to user goals, allergies, or concerns, and/or may exacerbate a disease or signs/symptoms of the disease. These personalized ingredient lists 104 then provide input to a product filter computer application 106 which, in turn, provides personalized product recommendations 108 of specific topical products matching results of the personalized ingredient lists. Topical products can include products relating to or associated with skin care, hair care, nail care, cosmetics, make-up, fragrances, personal care, OTC and prescription products.

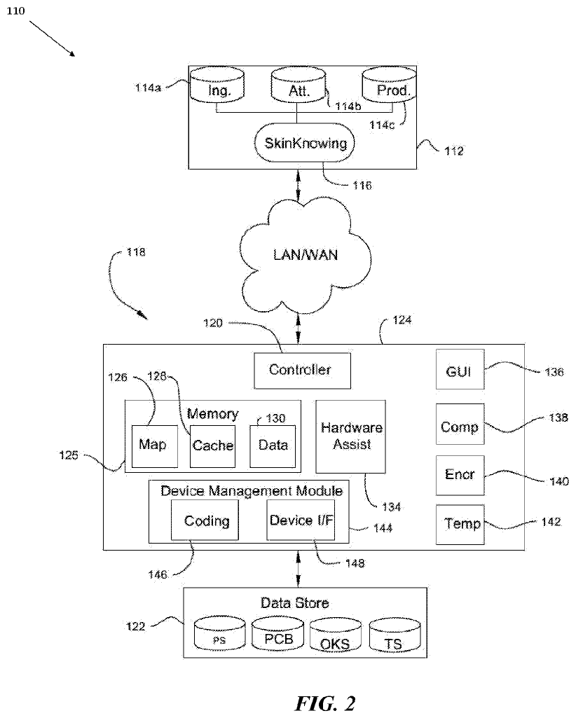

FIG. 2 schematically illustrates a skincare system 110 in accordance with illustrative embodiments of the claimed technology in the form of a cloud-based host 112 using information in a database 114 of ingredients, products, medical, genetic, chemical, biological, or cosmetic knowledge into a computer application 116. The computer application 116 may also be referred to herein as a SkinKnowing computer application 116. The computer application 116 may be configured to perform analysis or processing of user inputs and user attributes to determine ingredients and products that are recommended for use (therapeutic or okay to use) and not recommended for use. A user device 118 may be configured to communicate with the host 112 via a computer network connection. FIG. 2 is a generalized depiction of what the skilled artisan knows to be a wide variety of devices capable of executing cloud-based software, such as a desktop or laptop computer, cell phone device, camera, tablet device, and other like devices.

In these illustrative embodiments, the user device 118 includes a processor-based controller 120 which provides top-level control and communication functions as the user device 118 communicates with the host 112 to store and retrieve host user data. A memory module 122 provides non-volatile storage of the data, such as in the form of a hard disk drive (HDD), a solid-state drive (SSD), an array of flash memory cells, and the like. The controller 120 can be a programmable CPU processor that operates in conjunction with programming stored in a computer memory within the user device 118. The controller 120 can alternatively be a hardware controller. The controller 120 can be a separate circuit or the controller's functionality can be incorporated directly into the memory module 122.

As used herein, the term “controller” and the like will be broadly understood as an integrated circuit (IC) device or a group of interconnected IC devices that utilize a number of fundamental circuit elements such as but not limited to transistors, diodes, capacitors, resistors, inductors, waveguides, circuit paths, planes, printed circuit boards, memory elements, etc. to provide a functional circuit regardless of whether the circuit is programmable or not. The controller 120 can be arranged as a system on a chip (SOC) IC device, a programmable processor, a state machine, a hardware circuit, a portion of a read channel in a memory module, etc.

In more detail, illustrative embodiments of the user device 118 can be configured as an SSD that communicates with the host 112 via one or more Peripheral Component Interface Express (PCIe) ports. The non-volatile memory (NVM) can be NAND flash memory, although other forms of solid state NVM can be used. Flash memory control electronics can be provided to support parallel data transfer operations via a number of channels (lanes). The SSD can operate in accordance with the NVMe (Non-Volatile Memory Express) Standard. The systems and methods disclosed herein may be configured to operate compatibly with SaaS (Software as a Service) solutions for E-commerce retailors, systems in hospitals, and/or in-person or online clinics or pharmacies.

The user device 118 includes a controller circuit 124 in which the controller 120 maintains top-level control of all functions while generally performing host 112 interface functions and directing data transfers with the memory module 122. The controller 120 can have one or multiple programmable processors with associated programming (e.g., firmware, FW) in a suitable memory location, as well as various hardware elements to execute these front end, core, and back end data management and transfer functions. A pure hardware based controller configuration can alternatively be used. The various controllers can be integrated into a single system on a chip (SOC) integrated circuit device, or can be distributed among various discrete devices as required.

A controller memory 125 represents various forms of volatile and/or non-volatile memory (e.g., SRAM, DDR DRAM, flash, etc.) utilized as local memory by the controller 120. Various data structures and data sets can be stored by the memory 126 such as map structures 126, caches 128 for map data and other control information, and buffers 130 for temporarily storing host data during data transfers.

A non-processor based hardware assist circuit 134 can enable offloading of certain memory management tasks by one or more of the controllers, as required. The hardware circuit 134 does not necessarily utilize a programmable processor, but instead uses various forms of hardwired logic circuitry such as application specific integrated circuits (ASICs), gate logic circuits, filed programmable gate arrays (FPGAs), etc.

Other illustrative core function blocks can be used, such a customizable graphics-user interface (GUI) block 136, a data compression block 138, a data encryption block 140, a temperature sensor block 142, and the like.

A device management module (DMM) 144 supports back end processing operations. It can contain a coding circuit 146 for generating coding used in error detection and correction such as outer codes and low density parity check (LDPC) codes. The DMM 144 can also contain a device interface logic circuit 148.

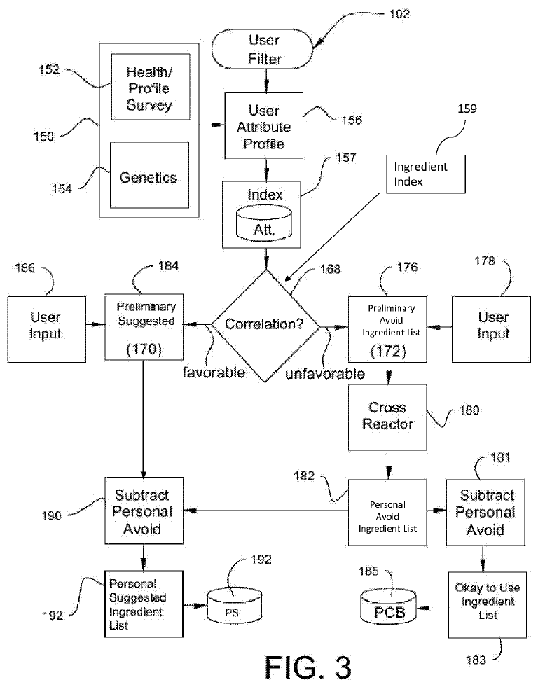

FIG. 3 schematically illustrates a flowchart depicting steps performed by the user device 118, in response to various external inputs, in performing illustrative embodiments of the user analysis 102 (FIG. 1) methodology. The method begins by providing personal information from the user in block 150. In these illustrative embodiments, some personal information is obtained by having the user complete a predefined health and profile survey 152. The survey 152 is designed to identify the individual user's risk factors, goals, and concerns. In some cases, the survey may be prepopulated partially or fully with information from another source such as an electronic medical record (EMR), one or more APIs, and the like. Also, the user personal information 150 in these illustrative embodiments includes the user's genetics information 154. The user's genetic information may be obtained and provided by having the user undergo commercially available genetics testing (e.g., through a company such as 23andMe or AncestryDNA), and then having the user (i) approve or provide access to that data through software system communication or API access or (ii) upload a raw data file comprising the user's genetic information to a server or platform that is configured to analyze the user's genetic information and generate or aid in the generation of one or more user attributes. The raw data file comprising the user's genetic information may be obtained through the commercially available genetics testing.

The user personal information 150 can be interpreted using a hidden complex analysis which considers (i) single answers/inputs, (ii) multiple answers/inputs in combination with one another, (iii) the pathogenesis, signs/symptoms, mechanisms involved for the conditions or attributes identified based on one or more user inputs, and/or (iv) risk and associated risks, in order to generate, populate, or define the user attributes profile in 156. Those user attributes in block 156 can arise from answers directly inputted by the user in 150 or from interpretation of one or more user attributes or user inputs provided by the user in block 150 (also called or referred to herein as inherited user attributes). The attributes in the user's profile block 156 may represent the user's individual risk factors, goals, and concerns that this skincare technology takes into consideration in rendering its ingredient/product recommendations. For example, if in answering the survey 152 the user states she has a disease (an inputted user attribute), then that disease's signs/symptoms, pathways, mechanisms, deficiencies, and/or risk factors can be defined or interpreted as inherited user attributes. Other inherited user attributes can comprise, for example, locations affected by the disease or condition, or locations showing one or more signs/systems of the disease or condition. Additional inherited user attributes can comprise, for example, risks for other disease states, or risks for other conditions or sensitivities to certain ingredients or products. Inherited user attributes can also arise from the interpretation of multiple inputted user attributes from block 150 and any relationships or interactions between the multiple user attributes (e.g., one user attribute mitigating or further exacerbating a condition associated with another user attribute). In some cases, combination of any of the user inputs described herein can be used to determine one or more inherited user attributes. For example, fair skin, sun exposure, and a history of basal cell skin cancer can be a user attribute independent of and distinct from just fair skin.

In some embodiments, user attributes may be determined based on survey answers (e.g., on health history, genetic, allergies, lifestyles, goals and concerns). The survey answers may be interpreted to determine genetic and health risks, symptoms and pathogenesis and associations of the user's goals, concerns and health problems, mechanisms that will exacerbate or ameliorate user's skin/hair/nail goals, and associated risks. Further, the survey answers may be analyzed to determine attributes beyond what is communicated by the user. For example, if a person has eczema on his or her arms, the systems described herein can determine that the person has risks for dry skin or irritated skin, increased risk for certain allergies, inflammation skin, itchy skin, etc., and interpret pathways of the skin that are overexpressed or that malfunction, such as decreased filaggrin and ceramides, altered skin pH, impaired barrier function and repair, altered buffered capacity, and/or increased inflammatory pathways. In another example, the presently disclosed systems can interpret combinations of answers to determine a user's attributes. For example, if a user just has high blood pressure versus a person who has high blood pressure plus high cholesterol, history of angina or heart attack risks can be determined differently and therefore product/ingredient recommendations may be interpreted differently for different users.

Some circumstances can cause any particular risk factor to be limited in some way. For example, in response to a user response indicating the user has glaucoma, the user attribute profile block 156 can be programmed to limit the recommended avoidance of products containing an unfavorable ingredient for glaucoma to those products that, when administered as directed, could possibly cause the purported unfavorable effect. That is, the user attribute block 156 can use the glaucoma attribute to recommend avoiding use of an eye cream or any product used around the eye containing the unfavorable ingredient but not necessarily avoiding use of a foot cream containing the unfavorable ingredient. Another example is that certain ingredients may only cause harm when formulated with other ingredients in the same product or when a user is using more than one product in the same area. Such user attributes that are at risk in certain circumstances related to product attributes (e.g., method or location of application or use), other ingredients, or other products and the like, may be referred to herein as special condition user attributes. When these special condition user attributes are run through the user filter 102 and result in one or more ingredients being included on the personal avoid list 182, they can be considered special condition avoid list ingredients.

Other predefined user attributes in 156 can be associated with risk factors stemming from something other than diseases. For purposes of this description of illustrative embodiments, the SkinKnowing application 116 can be assigned other attributes to the user corresponding to identified allergies, and corresponding to personal preferences/concerns for certain ingredients, be they health or environmental or moral preferences/concerns alike. In some cases, these user preferences/concerns can be measured or ranked on a scale to determine their level of concern/severity. Where the user lies on the scale may determine which ingredients should be avoided. In some cases, a user's cancer concerns can be stratified into discrete levels which correspond to differing levels of scientific evidence supporting a certain ingredient effect. For example, (1) known carcinogens—human data clinical trials, topical administration within concentrations used in beauty personal care, (2) potentially carcinogenic—in vitro data, animal data, oral admin or topically higher than what is found in products today, and (3) rumored carcinogens—mentioned to be carcinogenic without readily available or verifiable scientific evidence.

The user attribute profile block 156 can also be assigned other attributes corresponding to her genetics information 154. The uploaded raw data file contains information on the genes analyzed (SNP number) and the alleles present for each gene (G, C, A, T, Ins, or Del). The user attribute profile block 156 searches the raw data file for hundreds of genes that are targeted for potentially being a significant factor in determining which topical products the user should and should not be using. If the gene is present, then the user attribute profile block 156 looks further for a presence of a risk alleles for that gene. If the risk allele is present, then it is further determined whether one copy or two copies are present. The presence of one copy or two copies determines the relative risk (RR) or odds ratio (OR) of that particular genetic risk factor. If one copy of the risk allele is associated with significant (OR or RR) risk for disease for that particular gene, that triggers assigning the corresponding attribute. If two copies of the risk allele are present, or if a deletion or insertion of an allele is associated with significant risk for disease, that triggers assigning that corresponding attribute. In some cases, the presence of multiple genes and the presence of their corresponding or associated risk alleles can be noted and interpreted.

The user attribute profile block 156 identifies both inputted user attributes and inferred or inherited user attributes as described elsewhere herein by interpreting, analyzing, grouping, and referencing elements and/or combinations of elements of the user's personal information 150 to the clearinghouse information stored in user attribute database 114b. As described above, the inferred or inherited user attributes can be derived from a computer-based holistic interpretation of information that the user provided in a survey to determine user attributes beyond those directly communicated. In some embodiments, this clearinghouse repository of all possible user attributes can be established and maintained by the host. Alternatively, it can be established and maintained by the user, or cooperatively by both of them. In any event, the information on user attributes, inherited or inferred user attributes, or any other user attributes stored in the attribute database 114b, and any relevant information on ingredients that are (i) included in an ingredient index 159 or the ingredient database 114a and (ii) associated with the user attributes (e.g., either as recommended/okay to use/therapeutic ingredients or ingredients that are not recommended for use) can be gathered and continually updated and revised. In some cases, the user attribute information can be gathered from and updated based on additional user inputs received from the user or additional inferences derived from the user inputs or user attributes. In some cases, the ingredient information can be gathered from and updated based on published information, such as ingredient encyclopedias, medical research papers, product ingredient lists, and the like. Although FIG. 2 depicts this reference information stored in memory residing in the host 112, the contemplated embodiments are not so limited. In alternative embodiments this reference information can be stored in the user device, or elsewhere, or distributed thereabout. In some embodiments, the systems and methods described herein can be used to determine a list of ingredients in response to a user who says she is allergy prone but does not specifically know what she is allergic to, and/or to correlate various risk factors to different user ethnicities.

Also stored and maintained in host memory is a similar repository of all reference information on ingredients 114a that are commercially used in making topical products. Each ingredient listed in the database 114a can accompanied by other useful information called ingredient attributes such as aliases, cross reactors, ingredient families, chemical classes, sources, ingredient effects (both favorable and unfavorable), ingredient function in the topical product, an ingredient summary and bioavailability information, safety profiles, concentrations of use in products, ingredient interactions (e.g., ingredients that should not be used together), evidence based studies for the ingredient, a listing of which products include the ingredient, and the like. Ingredients, their relationships with other ingredients and their ingredient attributes in 114a can be gathered and continually updated and revised from published information, such as ingredient encyclopedias, medical research papers, product ingredient lists, and the like.

A similar reference database of all topical product knowledge can be established and maintained in a products database 114c (FIG. 2). Again, the illustrative embodiments depict this information stored in memory residing in the host 112, but the contemplated embodiments are not so limited. The product data is likewise gathered and maintained from published information, such as application programming interfaces (APIs) established with manufacturers and distributors of skincare products. The product data is merged from multiple sources to effectively differentiate any particular skincare product offering in terms such as product brand, name, sizes, counts, colors, scent or flavors. Preferably, the products database 114c provides, for each product listing, the product name(s), brand name(s), product sizes, product flavor(s), scent(s), color variation(s), universal product code (UPC), global trade item number (GTIN), Amazon standard identification number (ASIN), European article number (EAN), manufacturer part number (MPN), item number for each source, product image(s), product description, product categories, product consistency, product reviews, product location for use, product type, product price, product url, and ingredient list.

The information stored in the SkinKnowing engine 116 reflects reference knowledge of all known correlations that exist between each ingredient and ingredient attribute in the ingredient database 114a and each user attribute and inherited user attribute listed in the attribute database 114b and all product attributes in 114c. These relationships are gathered and continually updated and revised from published information, such as ingredient encyclopedias, medical research papers, product ingredient lists, and the like. The user filter 102 depicted in FIG. 3 can be configured to interpret and correlate user attributes and ingredients to determine specific ingredient recommendations for the user. This information also indicates the type of correlation, such as whether any particular correlation is a “favorable correlation” or an “unfavorable correlation.” As used herein, a correlation may refer to an association or relationship between one or more user attributes and one or more ingredients or ingredient attributes. In some cases, the correlation may comprise a 1:1 matching (e.g., if a user has a user attribute indicating that the user has eczema, then the correlation may comprise a negative association between the user's eczema and ingredients that may worsen the user's eczema). In other cases, the correlation may comprise a more complex association between user attributes or inherited user attributes and an ingredient or ingredient attribute that may affect or influence the user's attributes, and can extend beyond what was communicated by the user. For example, the systems and methods of the present disclosure may be used to determine that a user has eczema (user attribute), and inherited user attributes such as impaired skin barrier functions and impaired skin buffering capacity can also be assigned to the user, which means that the user may need to avoid ingredients that further impair skin barrier functions and/or further disrupt skin buffering capacity because this may worsen the user's eczema. Thus, combinations of user inputs/answers and the correlations between user attributes and ingredients may be created or identified based on (i) underlying or associated health conditions relating to one or more user attributes, and/or (ii) an understanding of a pathogenesis of the one or more user attributes and the like. As used herein, a pathogenesis may refer to any one or more biological mechanisms that may contribute to or affect a user attribute or a health condition associated with the user attribute. In some cases, the correlations may comprise a favorable correlation and/or an unfavorable correlation. A favorable correlation categorically defines an ingredient or group of ingredients to be acceptable for use for the corresponding attribute, or perhaps even better to be therapeutic, whereas an unfavorable correlation indicates the likelihood an ingredient or group of ingredients would potentiate the corresponding attribute. This information can also limit computations with an attribute, such as by further reflecting the strength of any particular correlation so that the user filter 102 (FIG. 1) and product filter 106 (FIG. 1) can compensate for conflicting or offsetting attributes. In product filter 106, a product attribute in 114c can also influence the negative or positive correlations between user attributes and ingredients. If a negative user attribute and ingredient attribute only exists for a certain method or location of use/application for a product, and the product being evaluated is not intended, manufactured or designed for that area of use, the negative correlation can be voided and deemed okay to use for the user profile.

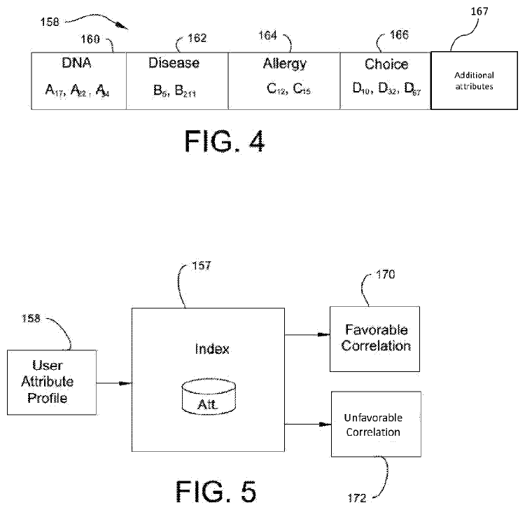

FIG. 4 schematically illustrates a user attribute profile 158 in accordance with illustrative embodiments, a data structure formed by the block 156 (FIG. 3) by referencing the attribute database 114b according to elements of the user's personal information 150. The data structure 158 can provide a personal expression of the user from various different perspectives, such as the user's risk factors, diseases, concerns, allergies, preferences, DNA, and the like and captures both inputted user attributes and inherited user attributes. For instance, FIG. 4 references the user attributes to reflect how they were gathered from different categories of the personal information 150, namely genetics information 160 (denoted “Aₙ”), disease information 162 (denoted “Bₙ”), allergy information 164 (denoted “Cₙ”), and information reflecting the user's personal preferences and concerns 166 (denoted “Dₙ”). In some cases, the user attributes may comprise additional attributes 167 such as lifestyle, goals/concerns (e.g., wellness concerns), age, ethnicity, allergy risks, or any inferred or interpreted attributes that are derived or determined based on multiple user inputs and/or correlations or interactions between other existing user attributes. For purposes of continuing the description of these simplified illustrative embodiments, in some non-limiting examples, the user's attribute profile 158 can comprise, for instance, three DNA attributes (A₁₇, A₂₂, A₃₄), two disease attributes (B₅, B₂₁₁), two allergy attributes (C₁₂, C₁₅), and three user's choice attributes (D₁₀, D₃₂, D₆₇). In some cases, the user attributes may be interpreted individually and/or in combination with one another in order to provide a personalized set of ingredient and/or product recommendations.

Referring to FIG. 2, FIG. 3, and FIG. 5, once the user attribute profile 158 is formed in block 156, then in block 157 the attribute database 114b is indexed according to the attributes collectively forming the user attribute profile 158. User attribute profile 158 can be based on single user attributes or a grouping or combination of user attributes. The grouping or combination of user attributes may comprise two or more user attributes that interact with each other or collectively influence a user's sensitivity or response to a certain chemical, material, ingredient, or product. Control then passes to block 168 which determines which, if any, of the ingredients in the database 114b are correlated to the user's attributes in her user attribute profile 158. This yields two subsets of the attribute database 114b, a set of favorably correlated ingredients 170 and a set of unfavorably correlated ingredients 172. In some cases, the correlations performed or interpreted in block 168 can be based on information received from an ingredient index 159 containing information on ingredients and various attributes, properties, or effects of the ingredients.

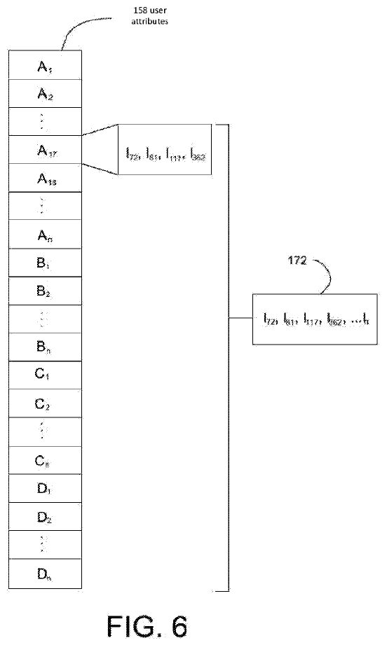

FIG. 6 schematically illustrates examples of an unfavorably correlated set of ingredients 172, consisting of or comprising all the ingredients that are unfavorably correlated to any of the user attributes in the user's attribute profile 158 as shown and described herein. For instance, the unfavorably correlated set 172 includes the four ingredients (I₇₂, I₈₁, I₁₁₇, I₃₆₂) that were determined to be unfavorably correlated to the DNA attribute A₁₇ in the user's attribute profile 158. The rest of the ingredients in the unfavorably correlated set 172 are those likewise unfavorably correlated to any of the other user attributes in the user's attribute profile 158. In some cases, the unfavorably correlated set of ingredients 172 can be determined or identified from groups or combinations of user attributes. In some cases, one or more algorithms may be used to interpret groupings of attributes (as opposed to single attributes) to determine which ingredients should be avoided. Such analysis may extend beyond a 1:1 correlation between ingredients and user attributes, and can include correlating multiple user attributes to one or more favorably or unfavorably correlated ingredients.

Referring back to FIG. 3, if the determination of block 168 is that there are unfavorably correlated ingredients for the user's attribute profile 158, then control passes to block 176 where the unfavorably correlated set 172 is referred to as the “Preliminary Avoid” ingredient list. It can be advantageous to give the user an opportunity to edit the Preliminary Avoid ingredient list in block 178, such as overriding the user filter 102 to manually add or delete ingredients, or changing responses to the survey 152, and the like. This allows the user to tune her user filter 102 to continually enhance its results.

Control then passes to block 180 where the user-edited Preliminary Avoid ingredient list is expanded to include cross reactors or chemically-similar ingredients, ingredient aliases, and the like. After all these changes, block 182 outputs a “Personal Avoid” ingredient list (also described herein as a final ingredient avoid list), which may comprise a personalized list of ingredients the user should personally avoid in making skincare purchases. In some cases, certain special condition avoid list ingredients that only need to be avoided in certain products and/or scenarios (e.g., application or use of the product in a particular location) can be included in an okay to use ingredient list for the user, or excluded from the personal avoid ingredient list. In block 181 the “Personal Avoid” ingredient list is subtracted from the entire list of ingredients under consideration in the ingredient database 114a to obtain the “Personal Clean Beauty” ingredient list (also referred to herein as an Okay to Use ingredient list) in block 183. A contemplated feature is that the GUI 136 (FIG. 2) is configured such that the user can conduct a query of whether a particular ingredient is OK for her to use. The “Personal Clean Beauty” ingredient list 183 provides a resource for the user filter 102 to reply to any such query with a straightforward “Yes” or “No” response. A “Yes” response will result if the queried ingredient is listed in the ingredient database 114a and in the Personal Clean Beauty (PCB) ingredient list 183. This means the ingredient may not negatively affect any user attribute or grouping of user attributes in 158. This PCB data set 185 is preferably stored in the user's device, such as depicted in FIG. 2 as being stored in the user device memory 122. A “No” response means that the ingredient may negatively impact a user attribute or group of user attributes assigned to the user profile in some way, and that it may be appropriate to include that ingredient on the Personal Avoid ingredient list 182.

If, otherwise, the determination of block 168 is that there are favorably correlated ingredients for the user's attribute profile 158, then control passes to block 184 where the favorably correlated set of ingredients 170 is referred to as the “Preliminary Suggested” ingredient list. As above, it can be advantageous to give the user an opportunity to iteratively tune the results of the user filter 102 by editing the Preliminary Suggested ingredient list in block 186, such as overriding the user filter 102 to manually add or delete ingredients, or changing responses to the survey 152, and the like. All ingredients of the Personal Avoid ingredient list of block 182 are subtracted from the user-edited Preliminary Suggested ingredient list 170 in block 190. After all these changes, block 192 outputs a Personal Suggested (PS) ingredient list, a personalized list of ingredients specifically recommended not just because they are OK to use, but because they may have therapeutic value or positive effects in relation to the user's attributes. These suggested therapeutic ingredients may only be relevant however under certain special conditions. For example, an ingredient that helps with acne would only be suggested or therapeutic in areas where the user has acne. Therefore, certain ingredients in a Personal Suggested (PS) ingredient list 192 can have designations as such but with special conditions, therefore titled a special condition suggested (or therapeutic) ingredient. This PS data set 192 is also preferably stored in the user's device, such as depicted in FIG. 2 as being stored in the user device memory 122.

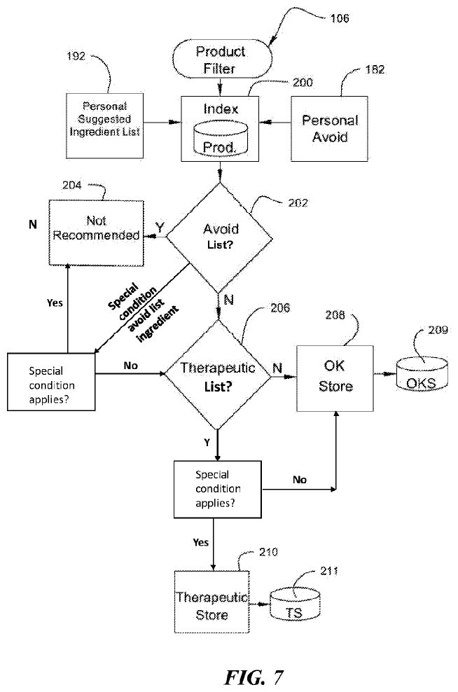

The process outputs of the user analysis 102 (FIG. 1), the PS ingredient list 192, the PCB ingredient list 185 (FIG. 1), and the Personal Avoid ingredient list 182 can be process inputs to the product filter 106 as depicted in the illustrative flowchart steps depicted in FIG. 7. In block 200 the ingredient list information for each product listed in the product database 114c is indexed to determine which products, if any, contain the ingredients of the three lists 182, 185, and 192. In block 202 the determination is made as to whether a listed skincare product contains one of the ingredients on the Personal Avoid ingredient list 182. If the determination of block 202 is “yes,” and that ingredient is not a special condition avoid list ingredient, that product is not recommended in block 204 for containing an ingredient that is adverse to the user's risk factors, and health concerns and goals. However if that ingredient is a special condition avoid list ingredient, then it must be checked to determine whether that special condition applies by looking at product attributes from 114c and the like (e.g., applied in a particular location or containing certain other ingredients or having a certain form or consistency). If the special condition is true, then the product is not recommended 204, and if it is false (i.e., not true or not applicable), then the product can be evaluated further to see if it is Okay to use or suggested/therapeutic in 206. If, otherwise, the determination of block 202 is “no,” then a further determination is made in block 206 as to whether the product has any ingredient on the PS ingredient list 192. If the determination of block 206 is “no,” then that product is recommended OK for use in block 208 because it does not contain any adverse ingredients. This OK products to use data set 209 is preferably stored in the user's device, such as depicted in FIG. 2 as being stored in the user device memory 122.

If the determination of block 206 is “yes,” then that product can be evaluated for any special conditions for that therapeutic condition such as special conditions relating to 114c product attributes and the like. If no special condition therapeutic ingredient exists then the product can be considered therapeutic and/or highly recommended (also referred to herein as a suggested product or a potential therapeutic product) in block 210 for its therapeutic value to the user's risk factors, goals and concerns. If the product comprises a special therapeutic ingredient, the product can be evaluated to see if a special condition (e.g., a relevant product attribute such as location of use/application or the like) is true. If the special condition is true then the product can be considered therapeutic (block 210). If not, then the product can be considered okay to use (block 208). The determinations in blocks 202 and 206 are made for all the products in the product database 114c, and the cumulative results of blocks 204, 208, and 210 produce one or more personalized product recommendations based on what ingredients and/or products the user should avoid, can use, and/or should use, respectively. The suggested products to use may be compiled in a data set 211, which may be stored in the user's device, such as depicted in FIG. 2 as being stored in the user device memory 122.

In another aspect, the present disclosure provides systems and methods for generating personalized ingredient lists and/or personalized product recommendations based on an in-depth analysis of one or more user attributes associated with a user or a consumer.

The systems and methods of the present disclosure may be implemented using a cloud software solution to revolutionize the way people shop for skin care, make up and personal care products. In some cases, the systems and methods of the present disclosure may be implemented using one or more knowledge graphs and/or neural networks. As used herein, a neural network may refer to a computational tool capable of machine learning. The neural network may comprise a plurality of interconnected computation units known as neurons that are configured to adapt to training data, and subsequently work together to produce predictions in a model that to some extent resembles processing in biological neural networks. The one or more neural networks may be used to generate one or more predictions or suggestions for recommended products to use and/or avoid, based on one or more user attributes associated with a user.

In some cases, the neural network may comprise a set of layers, the first layer being an input layer configured to receive an input. The input layer may comprise neurons that are connected to neurons associated with a second layer, which may be referred to as a hidden layer. Neurons of the hidden layer may be connected to a further hidden layer, or an output layer. The neural network may comprise, for example, fully connected layers and convolutional layers. A fully connected layer may comprise a layer wherein all neurons have connections to all neurons on an adjacent layer, such as, for example, a previous layer. In some cases, the neural network may comprise both fully connected layers and layers that are not fully connected.

In some cases, the neural network may comprise, for example, a deep neural network (DNN). In some embodiments, the deep neural network may comprise a convolutional neural network (CNN). The CNN may be, for example, U-Net, ImageNet, LeNet-5, AlexNet, ZFNet, GoogleNet, VGGNet, ResNet18, or ResNet, etc. In some cases, the neural network may be, for example, a deep feed forward neural network, a recurrent neural network (RNN), LSTM (Long Short Term Memory), GRU (Gated Recurrent Unit), Auto Encoder, variational autoencoder, adversarial autoencoder, denoising auto encoder, sparse auto encoder, boltzmann machine, RBM (Restricted BM), deep belief network, generative adversarial network (GAN), deep residual network, capsule network, or attention/transformer networks, etc. In some embodiments, the neural network may comprise a plurality of neural network layers. In some cases, the neural network may have at least about 2 to 1000 or more neural network layers.

In some cases, the systems and methods of the present disclosure may be implemented using one or more algorithms. The one or more algorithms may comprise a user analysis algorithm configured to determine what ingredients or combination of ingredients may cause users harm or provide users therapeutic benefits. The user analysis algorithm may be configured to determine what ingredients or combination of ingredients may (i) cause users harm or (ii) provide users therapeutic benefits, based on each user's genetics, skin type, health and/or skin history, allergies, consumer goals, and/or health concerns.

The one or more algorithms may further comprise a product analysis algorithm. The product analysis algorithm may be configured to assist users with identifying and selecting the cosmetic products that are right for them, and to avoid products that either cause the user harm or that are not compatible with the user's health concerns, genetic profile, consumer goals, and/or interests.

Applications

The systems and methods of the present disclosure may be used to inform users about which ingredients to avoid or use, based on their personal health or consumer goals, health concerns, allergies, DNA or genetic makeup, lifestyle, risk factors, health history, and/or skin/hair/nail concerns, goals, history, and/or type. The systems and methods of the present disclosure may be implemented to direct users to cosmetic products from major retailers that are compatible with one or more user attributes. Such user attributes may correspond to one or more personal, physical, psychological, physiological, mental, or genetic attributes associated with a user. The systems and methods of the present disclosure may be used to inform users if an ingredient or product is right for them or compatible with their user attributes by a simple “yes” or “no” answer. The systems and methods of the present disclosure may further provide evidence-based research behind every ingredient in our database. The systems and methods of the present disclosure may also be implemented to connect user to experts in the field for product recommendations and/or health care regimen advice, and may provide price comparisons between cosmetic or beauty products available from a plurality of different retailors. The systems and methods of the present disclosure may be implemented to provide a health, skin, and genetics analysis tool that analyzes user risk factors to determine which ingredients to use and/or avoid.

Algorithms

As described above, the systems and methods of the present disclosure may be implemented using a user analysis algorithm. The user analysis algorithm may be configured to determine what ingredients are recommended for a user, and what ingredients or combination of ingredients are not recommended for the user. Based on a user's ingredient recommendations, another algorithm (a product analysis algorithm) may be used to provide personalized product recommendations for the user.

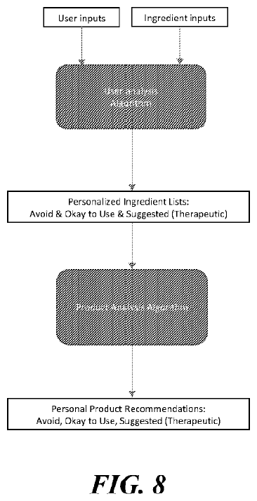

FIG. 8 illustrates a flow chart diagramming a process for generating personalized ingredient lists and personalized product recommendations. FIG. 8 shows a set of inputs and outputs of the user analysis algorithm and the product analysis algorithm described herein. In some cases, the user analysis algorithm may be configured to receive one or more user inputs (described in greater detail elsewhere herein). The user analysis algorithm may be configured to generate one or more personalized ingredient lists based on the user inputs. The personalized ingredient lists may be provided to another algorithm (e.g., a product analysis algorithm). The product analysis algorithm may be configured to use at least the one or more personalized ingredient lists generated using the user analysis algorithm to generate one or more personalized product recommendations for the user.

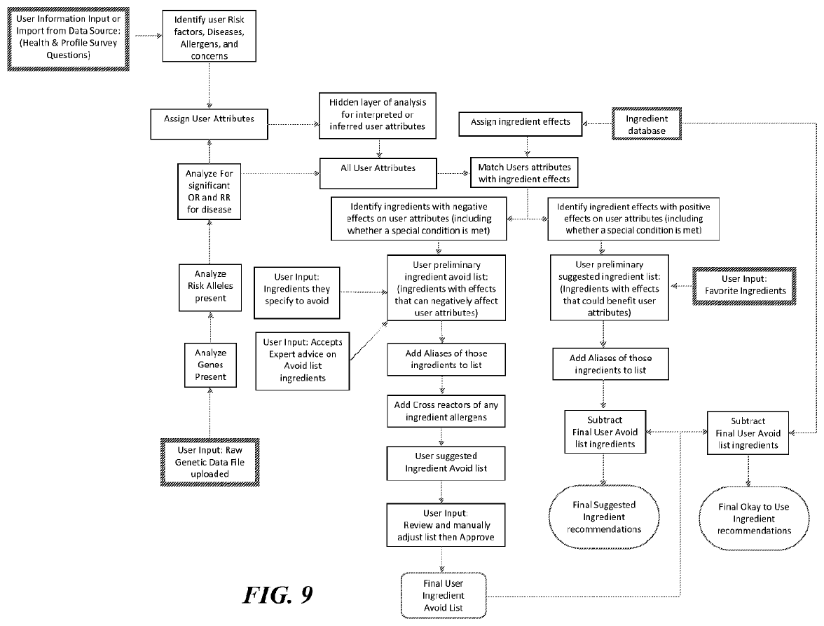

FIG. 9 illustrates a user analysis algorithm flow chart. The user analysis algorithm may be used to generate an ingredient avoid list, an okay to use ingredient list, and a therapeutically recommended or suggested ingredient list, based on one or more user-specific inputs or attributes (e.g., user risk factors, user health goals, user consumer goals, and/or user health concerns). Such inputs and/or attributes may be determined using a health survey and/or a user profile survey. In some cases, a first set of user attributes may be determined from various information gathered about the user or user inputs to a survey. The first set of user attributes may correspond to user risk factors, diseases, allergens, or concerns identified or inferred from the user inputs. In some embodiments, an additional layer of analysis may be implemented to analyze the user inputs collectively and holistically (thereby accounting for any interactions or relationships between various user inputs or attributes) in order to derive a second set of user attributes comprising various inferred user attributes (also referred to herein interchangeably as interpreted user attributes or inherited user attributes). The first set of user attributes and the second set of user attributes may be combined to form a comprehensive attribute profile for the user.

As shown in FIG. 9, the user analysis algorithm may be configured to assign user attributes based on one or more user inputs or information gathered about the user from outside data sources (e.g., EMRs and/or APIs). In some cases, the one or more user inputs may be provided through one or more health and profile survey questions. The one or more health and profile survey questions may prompt the user to review the information gathered directly from the user or from outside data sources, and/or input more information that may indicate or identify one or more user risk factors, diseases, allergens, health goals, and/or other health concerns. In some cases, the additional information input by the user may be used to confirm or verify one or more user risk factors, diseases, allergens, health goals, and/or other health concerns.

Survey Questions

As described above, the user analysis algorithm may be configured to generate recommended ingredient lists or ingredient avoid lists based on a user's inputs into a health and profile survey. Questions in the survey may ask about a user's age, ethnicity, sun exposure, general lifestyle habits, skin type, skin and health history, allergies, genetics, skin goals, and ingredient concerns and the like. The user analysis algorithm may be configured to generate recommended ingredient lists or ingredient avoid lists based on user inputs comprising information about ethnicity, skin, hair, and/or nail concerns, cosmetic and medical issues for skin, hair, and and/or nails, skin history, skin habits, skin risk factors, sun exposure, tanning booth, skin cancer, skin type, skin hydration, skin sensitivity, health history and/or family history with cancer, cardiac issues, or medical conditions associated with a lung, an ear, a nose, or a throat, gastrointestinal issues, neurological conditions, endocrine disorders, hematologic disorders, rheumatological disorders, ocular disorders, or any other type of medical condition. In some cases, the user analysis algorithm may be configured to generate recommended ingredient lists or ingredient avoid lists based on user inputs comprising information about allergies (environmental, drug, food, cosmetics, specific ingredients) and/or product and ingredient concerns pertaining to, for example, the environment, cancer, toxicity, neurotoxicity, reproductive and developmental toxicity, inflammatory risks, allergenic risks, hormonal influence, sustainability, and/or whether an ingredient is banned by the FDA or other countries outside the U.S.

By way of example, if a user inputs one or more answers to a survey question, the user analysis algorithm may be configured to stratify the user's answers as a risk or not a risk, and may then add the information to the user's profile as a user attribute. For example, if the user has a goal of reducing pigmentation on their face, the user analysis algorithm may help the user to avoid ingredient in facial products which may increase pigmentation. In another example, the user may be asked about how much skin irritation the user experiences from products use. If the user says all the time, then the risk for skin irritation would we added as a user attribute. If the user responds that he or she never experiences skin irritation, then this would be added as a user attribute but not be stratified as a risk. Or if the user designates their age as 6 months, the user analysis algorithm may be configured to designate the user as a baby and assign one or more user attribute risks based on the user's age.

In some cases, the user analysis algorithm may also account for user attributes that correspond specifically to one or more locations or portions on the user's body or other unique conditions that are referred to herein as special conditions. For example, if a user indicates that they have the eye disease glaucoma, then the user analysis algorithm can classify that user attribute as a risk associated with the user's eye area or region. Or if a user experiences thinning hair on his or her head, the user analysis algorithm can classify that user attribute only with respect to the hair and scalp regions of the user's body. The user analysis algorithm can also determine or anticipate whether a product contains ingredients that can affect one or more user attributes when used together, or whether multiple products can cause an effect on one or more user attribute when used together.

In some embodiments, the user attributes may be analyzed against and/or matched to ingredient effects in order to (1) identify ingredients with negative effects on user attributes (depending on whether a special condition is met), and (2) identify ingredient effects with positive effects on user attributes (depending on whether a special condition is met). The special condition may comprise, for example, a location in which the ingredient or product is used or applied, a method of applying or using the ingredient or product, a physical form of the ingredient or product, or the like.

In some cases, the user analysis algorithm may be configured to receive one or more user inputs associated with a user's generalized concerns about one or more ingredients and/or products. Such user inputs may be processed to generate another set of user attributes. In some cases, various groupings of user inputs or user attributes may be further analyzed to derive one or more interpreted or inferred user attributes. The first set of user attributes derived from survey inputs and the second set of user attributes derived by analyzing groupings of user inputs or other user attributes may be aggregated to define all of the user attributes for a particular user.

In some cases, the user may be asked to quantify and/or grade their general ingredient and product concerns on a qualitative or quantitative scale. Different user attributes may be assigned depending on how a user grades and/or quantifies their generalized concerns or the level of evidence they require to substantiate their concern. For example, if a user is only slightly concerned about inflammatory ingredients and products, then a user attribute may be recorded as such and only ingredients that are very inflammatory and have strong scientific evidence of their inflammation may be assigned to the user's avoid list. Ingredients having strong supporting scientific evidence may comprise, for example, ingredients with topical human studies showing a beneficial effect within the concentrations found in commercially available topical products. On the other hand, if a user is severely worried about carcinogenic ingredient, a corresponding user attribute may be recorded as such, and any ingredient with an association with carcinogenic mechanism in any type of study may be assigned to the user's ingredient avoid list. Such study may include, for example, scientific evidence derived or obtained from in vitro studies, animal studies, topical high concentration studies, or studies using other non-topical routes of administration, and the like. Additionally, if a user indicates that he or she is slightly concerned about inflammation, then the user may be assigned a user attribute that would permit the user analysis algorithm and/or the product analysis algorithm to suggest ingredients or products with some evidentiary support or clinical evidence showing potential inflammatory effects (including, for instance, ingredients with rumored effects, wherein such rumored effects have been mentioned in news/blogs, etc. but do not have readily accessible or verifiable supporting evidence, or are mentioned in studies that do not affirmatively support the effects, or are not corroborated or debunked by any studies at all due to an absence of relevant studies). If the user indicates that he or she is severely concerned about carcinogens, then the user may be assigned a user attribute that would permit the user analysis algorithm and/or the product analysis algorithm to suggest ingredients or products with substantial evidentiary support or clinical evidence of reduced or minimal carcinogenic effects.

In some cases, a user may provide information pertaining to the user's allergies or concerns about allergic reactions. If a user inputs allergies, one or more corresponding risk factors may be added to the user profile as a user attribute. For example, if a user indicates that they are allergic to sulfa medications, the user analysis algorithm may be configured to add all sulfa ingredients to the user's ingredient avoid list on account of the user's allergy. If a user indicates that they are allergic to a specific ingredient or family of ingredients, the user analysis algorithm may be configured to add those specific allergies to the user's profile as a user attribute, along with any ingredients with any cross reacting properties. As used herein, an ingredients with cross reacting properties may refer to any ingredient with a similar chemical structure proven to also cause allergic reactions for users who are allergic to a specific ingredient. Users who have many allergies to products may provide information about their allergies in the survey. Users who do not know what they are specifically allergic too may have the option to add the most common ingredient allergens in cosmetics and personal care to their avoid list. Such information on ingredient allergy frequencies may be derived from a combination of patch testing allergy data, data from various information databases, and/or data from one or more dermatology practices.

In some cases, a user may also specify if they want to avoid any particular ingredients or family of ingredients for any reason. When they do this, those ingredients may be added to the user's personalized avoid list.

In some embodiments, the user analysis algorithm may be configured to match one or more user attributes with one or more ingredient attributes. Such ingredient attributes may be associated with one or more ingredients that are input into an ingredient database. The ingredients within the ingredient database may be gathered from product ingredient lists, and may be merged to account for different spellings and/or chemical names that may be listed as roots or aliases. Each ingredient in the ingredient database may also be assigned and/or associated with various cross reactors, ingredient families, chemical classes, sources, ingredient effects (both positive and negative), functions, formulas, and/or products that contain each ingredient. In some cases, write up summarizing what is known about the ingredient may also be provided to the user. Any information associated with an ingredient may be assigned to the ingredient as an ingredient attribute. Ingredient properties may include, for example, aliases (other names), chemical cross-reactors, ingredient family, chemical class, derived from sources, ingredient effects (positive and negative), function in the formula, and/or products that contain these ingredients.

As shown in FIG. 9, the user analysis algorithm may be configured to match or associate one or more user attributes to one or more ingredient attributes that may potentiate a user's risk factors or disease, cause an allergic reaction, or that are counter-intuitive to the user's health concerns or wishes. In some cases, the user analysis algorithm may be configured to match ingredients with a negative effect on one or more user attributes, and to generate a user preliminary ingredient avoid list. The user preliminary ingredient avoid list may comprise one or more ingredients with effects that may negatively potentiate a user's attributes. Sometimes these negative effects can only happen in special conditions (e.g., when used in a certain area of the body, when used with certain other products or ingredients, when used by consumers with certain genetics, and the like). These special condition can be associated with the ingredients assigned to certain specific user attributes. The user analysis algorithm may be configured to add aliases of those ingredients to list, add cross reactors of any ingredient allergens, and generate a user suggested ingredient avoid list. The user analysis algorithm may be configured to receive a user input whereby the user can review, manually adjust, and approve the user suggested ingredient avoid list or a modification thereof. This may generate a final ingredient avoid list. In some cases, the user analysis algorithm may be further configured to match user attributes with one or more ingredient with a therapeutic or positive effects. The user analysis algorithm may then be configured to generate a user preliminary suggested ingredient list, which may comprise ingredients with effects that could benefit one or more user attributes. In some cases, special conditions relating to a method or location of application or use may be analyzed to determine whether the ingredient or product is okay to use, recommended, or not recommended (to be avoided). The user preliminary suggested ingredient list may be aggregated and/or modified based on a user input corresponding to the user's favorite ingredients. The user analysis algorithm may be configured to then add aliases of those ingredients to a list of ingredients, and then subtract ingredients appearing in the final user ingredient avoid list to generate a listing of suggested ingredient recommendations.

As described above, in some cases the user's preliminary avoid list ingredients may be expanded to include all ingredient aliases and any chemically similar ingredient that could cause an allergic reaction. Such chemically similar ingredients that could cause a similar allergic reaction may be referred to herein as ‘cross reactors.’ This new list may become the user's suggested avoid list. The user's suggested avoid list may then be displayed for the user. The user may modify and/or update his or her avoid list by manually deleting any ingredient or family or ingredients from the list, changing his or her answers in the survey, or manually adding an ingredient to the list. Once this list is approved by the user, it may be considered the final user avoid list.

In some cases, the user analysis algorithm may be configured to generate an ‘Okay to use’ ingredient list and/or a ‘suggested’ ingredient list. An additional ingredient list may also be assigned to the user, and this list may be called the ‘okay to use’ list and the ‘suggested’ or ‘therapeutic’ ingredient list. The ‘okay to use’ list may be created by taking all of the ingredients in the ingredient database and subtracting the ‘final avoid list’. The ‘suggested’ list may be generated by taking the user's attributes and matching them to ingredient attributes that will help reduce the users risk factors, disease, allergies, or concerns, and benefit their goals. This list may be referred to herein as a preliminary suggested or therapeutic ingredient list. All final user avoid list ingredients may be subtracted from this preliminary suggested ingredient list to form the final user ‘suggested’ or ‘therapeutic’ ingredient list. Any special conditions for ingredients to be avoided, or other ingredients deemed to be okay to use or suggested/therapeutic, can be analyzed, notated, and communicated to the user.

Genetic Analysis

In some cases, the one or more user inputs may further comprise a raw genetic data file upload. The raw genetic data file may be used to analyze a user's DNA (genetics). The raw genetic data file may comprise information on one or more genes, one or more single nucleotide polymorphisms (SNPs), and/or one or more alleles associated with one or more genes (e.g., insertion alleles, deletion alleles, and/or alleles comprising one or more modifications or mutations pertaining to one or more portions or bases of a nucleotide sequence, which bases may comprise guanine, cytosine, adenine, thymine, and/or uracil). The raw genetic data file may be processed or searched to identify one or more specific genes of interest, which genes of interest may or may not indicate a predisposition for a particular health condition or risk. In some cases, the user analysis algorithm may be configured to process and/or search the raw genetic data file to identify one or more SNPs.

Referring back to FIG. 9, if one or more genes of interest are present in the raw genetic data file, the user analysis algorithm may be configured to look for the presence of one or more risk alleles associated with the one or more genes of interest. If a risk allele is detected, the user analysis algorithm may be configured to determine if there is one copy or two copies of the risk allele are present. The presence of one copy or two copies determines the relative risk (RR) or the odds ratio (OR) of a particular genetic risk factor associated with the risk allele. If one copy of the risk allele is associated with a significant (OR and/or RR) risk for disease for that particular gene, the user analysis algorithm may be configured to add that risk factor to a user profile as a user attribute. If two copies of the risk allele or a deletion or insertion of an allele is associated with a significant risk for a disease, the user analysis algorithm may be configured to add that risk to the user profile as a user attribute. The user analysis algorithm may also allow for user attributes to be added only if multiple genes have a certain risk allele, and in some cases, the associated user attributes may only be deemed significant for certain ethnicities or certain parts of a user's body. For example, if a user has a genetic risk for glaucoma, the user attribute would only be significant for cosmetic products used on the eye lashes or around the eye, and may not be significant for products used elsewhere on the user's body. Another example of user attribute determined by genetics is the gene for Age related Macular degeneration (ARMD). Those with risk alleles for this gene may have increased glycation and may experience a formation of age related glycation end (AGE) products (i.e., modifications of proteins or lipids that become nonenzymatically glycated and oxidized after contact with aldose sugars). This may be relevant to other diseases such as diabetes and osteoarthritis, as well as health conditions associated with the ageing of the skin, including skin sallowness. If a user has this risk genetically, it is important to avoid any ingredients that promote glycation, and it would be beneficial for the user's skin if the user uses products with ingredients that can prevent such AGE products. In sum, all of a user's risk factors, diseases, concerns, allergies, and wishes may be collected through a survey and/or through genetic analysis and may be added to that user's profile as user attributes.

In some cases, the systems and methods of the present disclosure may be configured to implement a product analysis algorithm. Product data pertaining to one or more beauty or cosmetic products may be gathered from multiple API's and in some cases may be manually inputted. The product data may be merged from various sources so that the same product sold on multiple sites may be listed in the database as one product and all product variations such as different sizes, product counts, colors, scent or flavors may also be associated with the same product listing. For each product, the product data may comprise, for example, product name, brand name, product sizes, product flavor, scent or color variations, UPC, MPN, EAN, ASIN, item number for each source, product images, product descriptions, product categories, product consistency, product reviews, product location for use, product type by problem, product price, product URL (for sites sold) and an ingredient list comprising one or more active ingredients and/or one or more inactive ingredients.

The product analysis algorithm may be configured to analyze the outputs from the user analysis algorithm (i.e. the final user ingredient avoid list and the final therapeutic ingredient list with any special condition requirements) along with the product data to provide the user personalized product recommendations. The product analysis algorithm may be further configured to inform each user about what products are right for the user and which products are not right for the user, based on what ingredients are on the user's personalized lists. The product analysis algorithm may be further configured to tell a user if a product is not recommended, is recommended, or is a therapeutic suggested product.

Ingredients that are not recommended may be colored red or orange to signify whether they should be avoided for health and wellness reasons or allergy reasons, respectively, and may be accompanied with a red thumbs down. In some instances, with the thumbs up or down icons, there may also be text explaining why the ingredient is on the users avoid list or not recommended. Ingredient that are okay to use or therapeutically recommended may be accompanied with a thumbs up in a blue/green color. Ingredient can also be designated as favorites by the user. If an ingredient is marked as a favorite, it may be taken off the user's avoid list as a rule.

As described above, ingredients and/or products may be designated as recommended or not recommended. ‘Not recommended’ (thumbs down) may indicate that a product contains one or more ingredients on the user's final ingredients avoid list that are counterintuitive to the user's risk factors, health, allergies, concerns or goals. ‘Recommended’ (thumbs up) may indicate that a product does not contain any ingredients on the user's final ingredient avoid list that are counterintuitive to their risk factors, health concerns or goals. In some cases, one or more ingredients and/or products may be designated as a ‘therapeutic suggested product’ (thumbs up), which may indicate a recommended product that also contains ingredients that appear on the final user therapeutic ingredient list and that may be of value to the user's risk factors, diseases, goals or concerns. Alternatively, this product may also have clinical studies supporting its therapeutic value to the user's risk factors, diseases, goals or concerns. The final output of the product analysis algorithm may comprise personalized product recommendations provided to a user. The personalized product recommendations may comprise products that are recommended or recommended with an extra designation if the product has a therapeutic value. The personalized product recommendations may exclude products that are not recommended.

In some embodiments, an ingredient can be on the avoid list but not “thumbs down” in a product if that product does not contain attributes that put the user at risk. For example, if an ingredient is a special condition avoid list ingredient that is to be avoided only when used around the eye, a foot product with that ingredient would not be “thumbs down” for the user (i.e., the product having the ingredient in question may still be deemed okay to use or recommended for use).

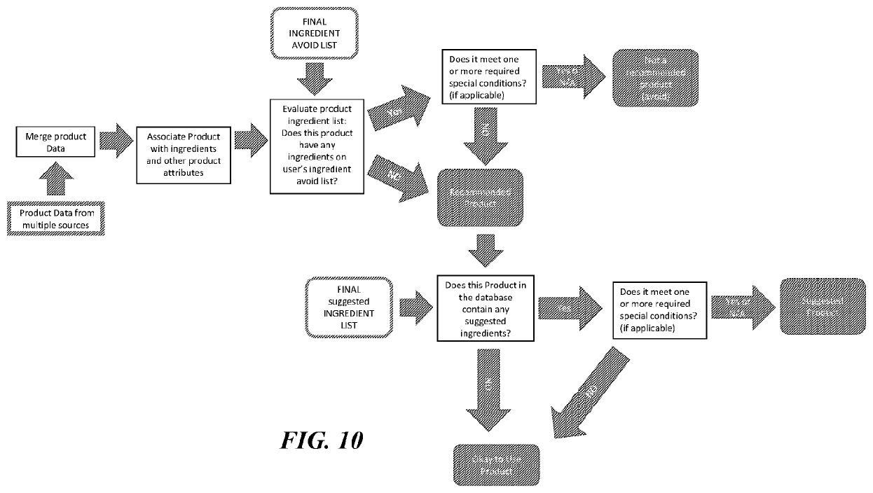

FIG. 10 illustrates a product analysis algorithm flow chart. The product analysis algorithm may be configured to receive product data from multiple sources and to merge such product data. The product analysis algorithm may be further configured to associate one or more beauty or cosmetic products with one or more ingredients. The product analysis algorithm may be configured to then evaluate a compiled list of beauty or cosmetic products against a user's final ingredient avoid list, final okay to use ingredient list, and/or final suggested ingredient list. As described elsewhere herein, the Final Ingredient Avoid List, the Final Okay to Use Ingredient List, and the Final Suggested or Therapeutic Ingredient List may be outputs from the user analysis algorithm. The product analysis algorithm may be configured to determine if one or more beauty or cosmetic products in the compiled list of beauty or cosmetic products have any ingredients that appear on the user's ingredient avoid list. If one or more beauty or cosmetic products in the compiled list of beauty or cosmetic products have one or more ingredients that appear on the user's ingredient avoid list (and said one or more ingredients (i) are special condition avoid list ingredients that (ii) meet one or more special conditions associated with, for example, method or location of application), the product analysis algorithm may be configured to identify such beauty or cosmetic products as products that are not recommended for a user (i.e., products that the user should avoid). On the other hand, if one or more beauty or cosmetic products in the compiled list of beauty or cosmetic products do not have any ingredients that appear on the user's ingredient avoid list, the product analysis algorithm may be configured to identify such beauty or cosmetic products as products that may be recommended to the user (i.e., okay to use, or suggested as a potential therapeutic product, e.g., if a special condition is met). The product analysis algorithm may be configured to determine if such beauty or cosmetic products contain any suggested ingredients that appear in the user's final suggested ingredient list. If so, the product analysis algorithm may be configured to identify such beauty or cosmetic products as suggested products or potential therapeutic products. If not, the product analysis algorithm may be configured to identify such beauty or cosmetic products as recommended products or products that are okay to use, even though the products may not necessarily provide any therapeutic benefits.

In any of the embodiments described herein, the user analysis algorithm and/or the product analysis algorithm may be configured to generate one or more ingredient lists or product lists (e.g., a Final User Ingredient Avoid List, a Final Okay to Use Ingredient List, a Final User Suggested or Therapeutic Ingredient List, a Recommended or Okay to Use Product List, a Suggested or Potential Therapeutic Product List, and/or a Product Avoid List) based on one or more ingredient or product attributes, also called special conditions. The one or more ingredient or product attributes (or special conditions) may comprise, for example, a form of application (e.g., an oil, a cream, an emulsion, etc.) or a location of application or use (e.g., a user's eyes, face, body, etc.). In one example, if a user has one or more attributes indicating that the user may have glaucoma (or that the user is susceptible to glaucoma), the user analysis algorithm and/or the product analysis algorithm may be configured to categorize or list one or more ingredients or products as ones that the user should avoid if such ingredients or products are applied on or near an eye of the user.

User Interface

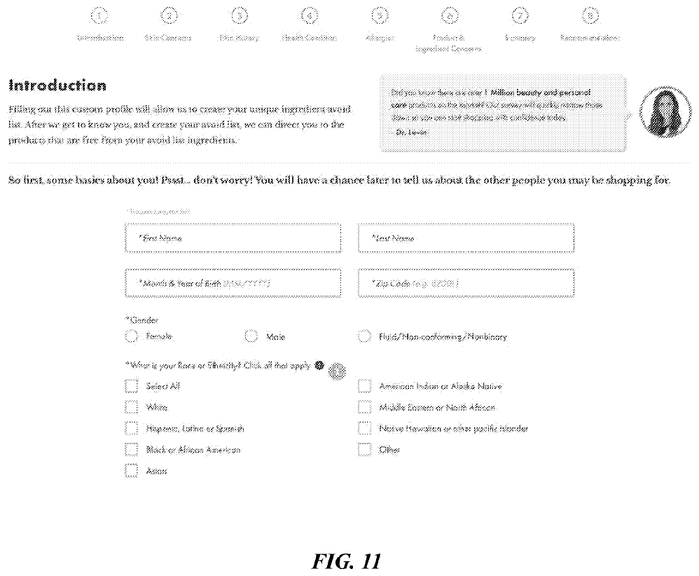

In some cases, the systems and methods of the present disclosure may be implemented through a user interface or a graphical user interface (referred to herein as a GUI or UI). As shown in FIG. 11, the UI may be configured to walk a user through a series of steps for inputting or reviewing gathered information about the user (e.g., the user's age, gender, ethnicity, etc.), skin concerns, skin history, health conditions, allergies, and/or product and ingredient concerns and the like. The UI may be configured to present the user with one or more recommendations for products or ingredients based on the inputs provided by or displayed to the user. In some cases, the inputs provided by the user or gathered from another source or API may correspond to one or more common allergies. As used herein, a common allergy may refer to an allergy that commonly occurs among a subset of a population. If a user is unsure about which specific allergies the user may have, the user may indicate to the UI or the platform implementing the UI that the user would like to receive one or more recommendations for products or ingredients that do not trigger or enable such common allergies. In any of the embodiments described herein, at least a portion of the information in the UI may be prepopulated based on information gathered from accessing another software system or API and the like. In some cases, at least a portion of the information in the UI may be prepopulated based on information from an electronic record (e.g., an electronic medical record or an electronic health record).

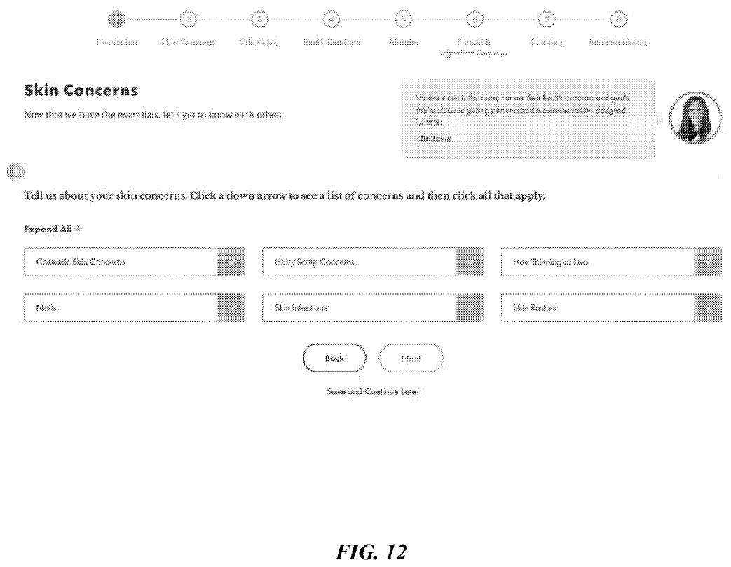

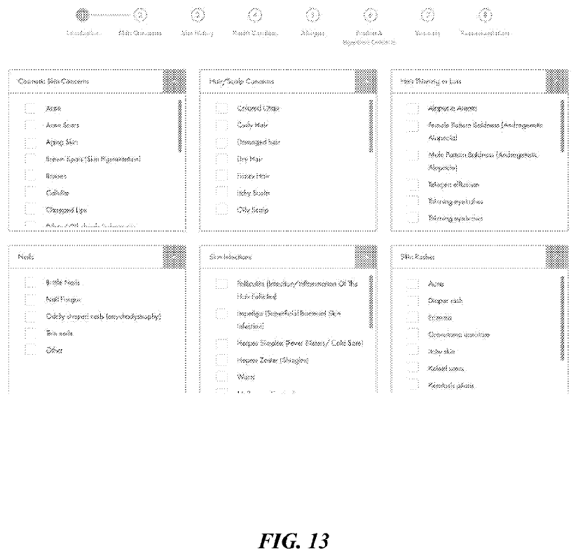

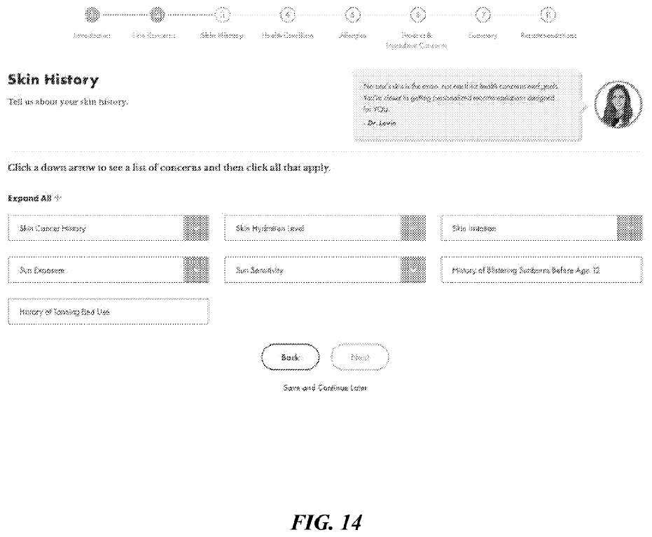

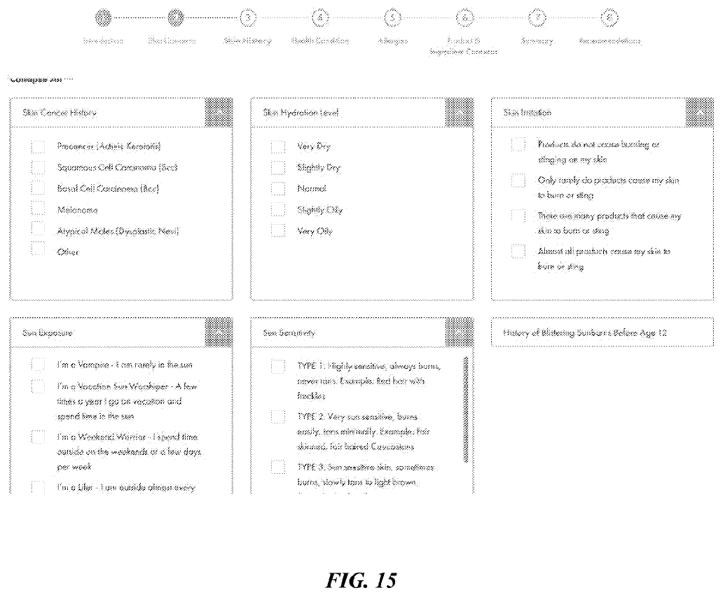

The UI may prompt the user to enter information about one or more skin concerns that the user may have (FIG. 12). The UI may also display or prompt the user for information about cosmetic skin concerns, hair and/or scalp concerns, hair thinning or loss, nails, skin infections, and/or skin rashes and the relevant location(s) of these concerns/goals. (FIG. 13). In some cases, the UI may display or prompt the user for information about the user's skin history (FIG. 14). The UI may also display or prompt the user for information about skin cancer history, skin hydration level, skin irritation, sun exposure, sun sensitivity, and/or a history of blistering sunburns before a certain age (FIG. 15).

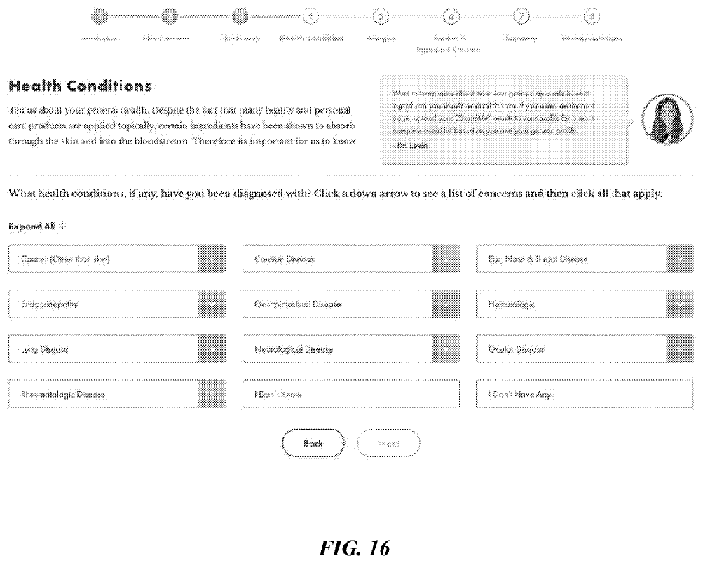

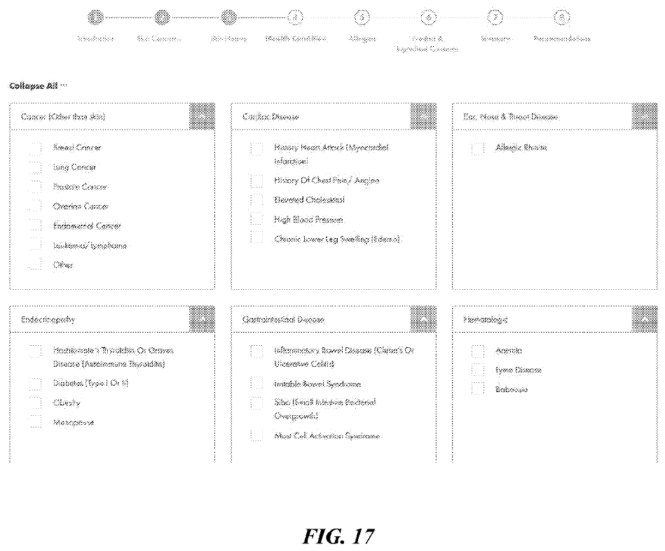

In some embodiments, the UI may display gathered information or prompt the user to enter information about one or more health conditions (FIG. 16). The one or more health conditions may comprise, for example, cancer, cardiac disease, ear, nose, and throat diseases, endocrinopathy, reproductive history, gastrointestinal diseases, hematological disorders, lung disease, neurological disease, ocular diseases, and/or rheumatological diseases. The UI may be configured to present the user with one or more collapsible drop down menus to review and/or select additional conditions and/or medical disorders pertinent to the user (FIG. 17).

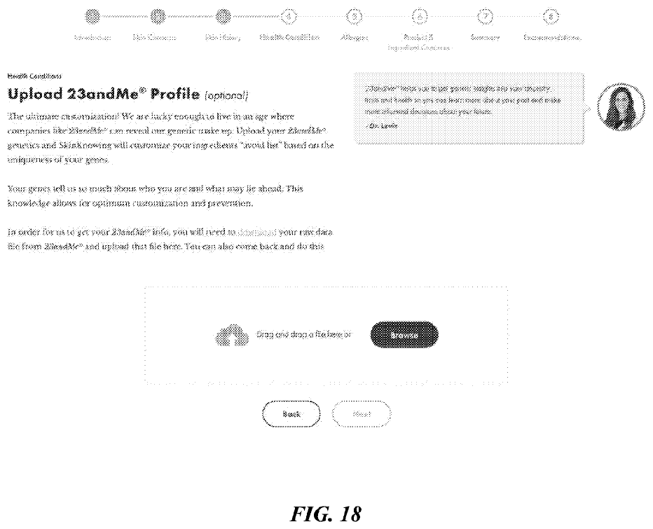

In some embodiments, the UI may prompt the user to enter the user's genetic information, which may be stored in a raw genetic file (FIG. 18). The raw genetic file may be generated based on an analysis of a biological sample of the user or gathered from another software system or API. The genetic information stored in the raw genetic file may be used to generate one or more user attributes as described elsewhere herein.

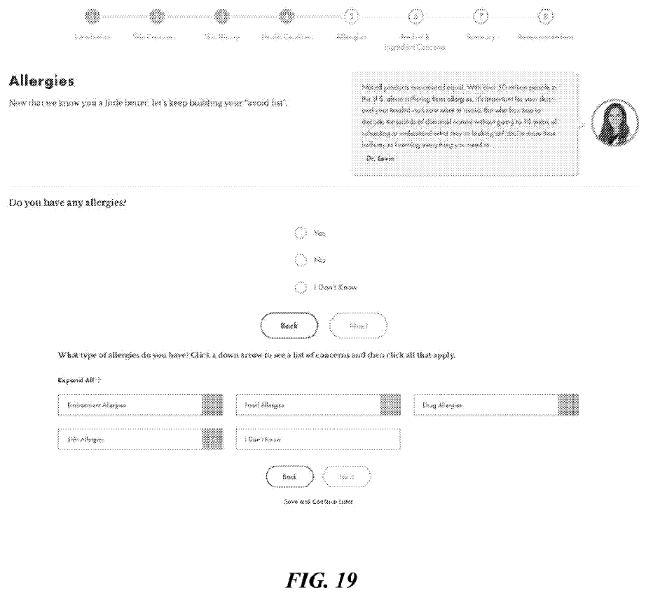

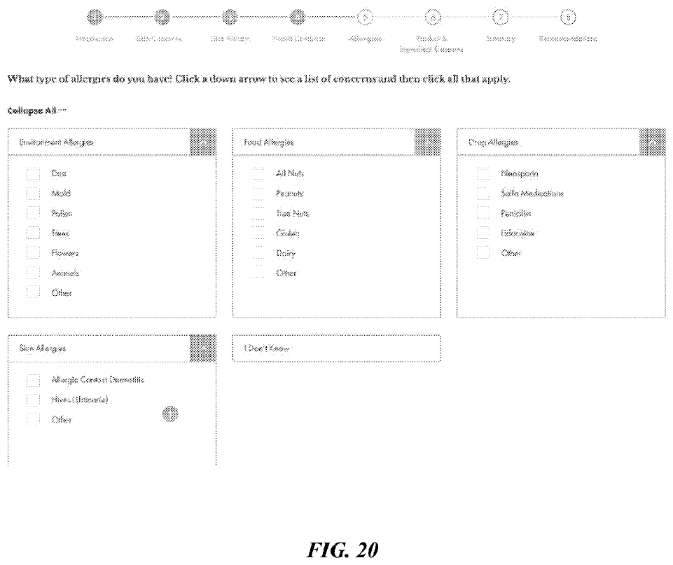

In some embodiments, the UI may prompt the user to review and/or enter information about one or more allergies that the user may have (FIG. 19). The one or more allergies may be, for example, environmental allergies, food allergies, drug allergies, and/or skin allergies (FIG. 20).

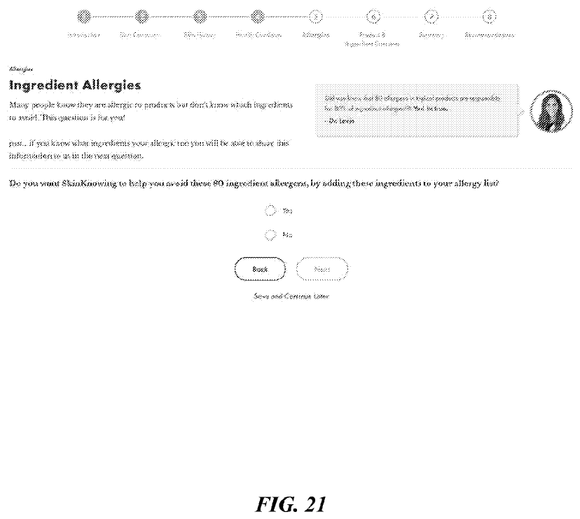

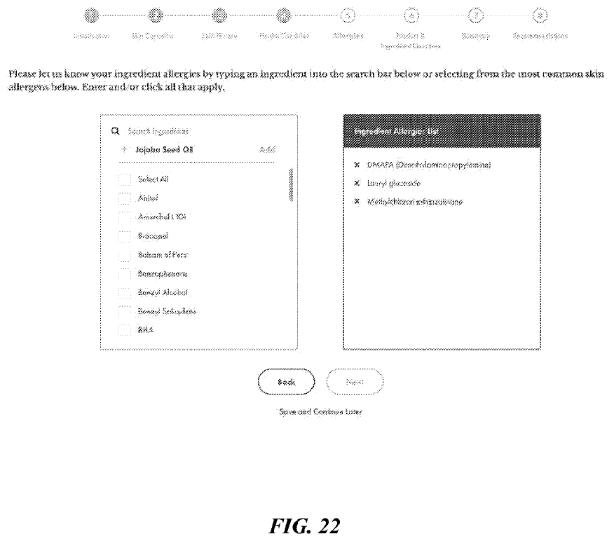

In some embodiments, the UI may display or prompt the user to enter information about one or more ingredient allergies (FIG. 21). As shown in FIG. 22, the UI may be configured to permit the user to search for and select one or more allergies or allergens from a list or database of common allergies and allergens. The UI may also permit the user to generate a preliminary ingredient allergies list.

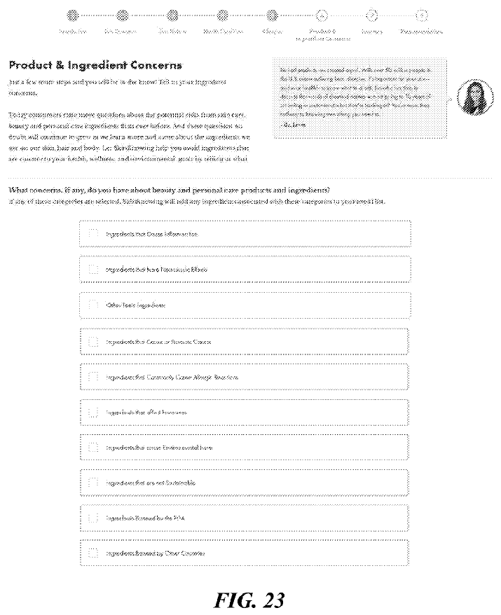

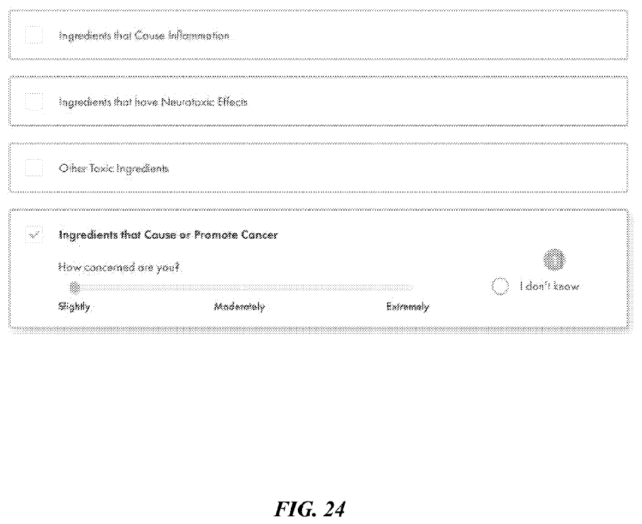

The UI may also permit the user to review and/or enter information about one or more product or ingredient concerns (FIG. 23). The product or ingredient concerns may comprise, for example, concerns about ingredients that cause inflammation, ingredients with neurotoxic effects, toxic ingredients, ingredients that cause or promote cancer, ingredient that commonly cause allergic reactions, ingredients that affect hormones, ingredients that cause environmental harm, ingredients that are not sustainable, ingredients banned by the FDA, and/or ingredients banned by one or more states, countries, counties, or regions. As shown in FIG. 24, the UI may provide the user with a sliding scale that the user can manipulate to indicate a level of concern associated with a particular ingredient or product (e.g., slightly, moderately, extremely, and the like).

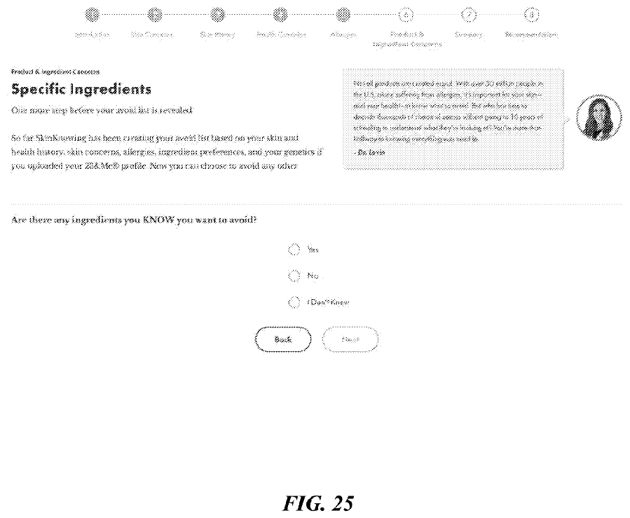

In some embodiments, the UI may further prompt the user to review and/or enter information about one or more specific ingredients or products that the user would like to avoid (FIG. 25). In some cases, the UI may be configured to generate a list of products or ingredients for the user to avoid, based on one or more ingredients or products that the user indicates as ingredients or products that the user would like to avoid.

In some embodiments the UI may further prompt the user to review or enter information about their favorite ingredients and products in order to make additional ingredient and product recommendations. In some embodiments, the UI may also prompt the user to review or detail their topical regimens by location of use and time of application and order of application.

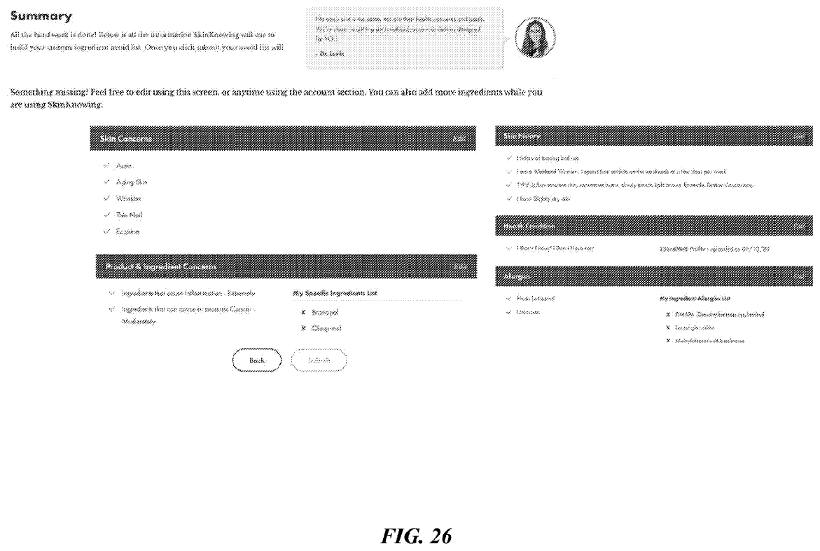

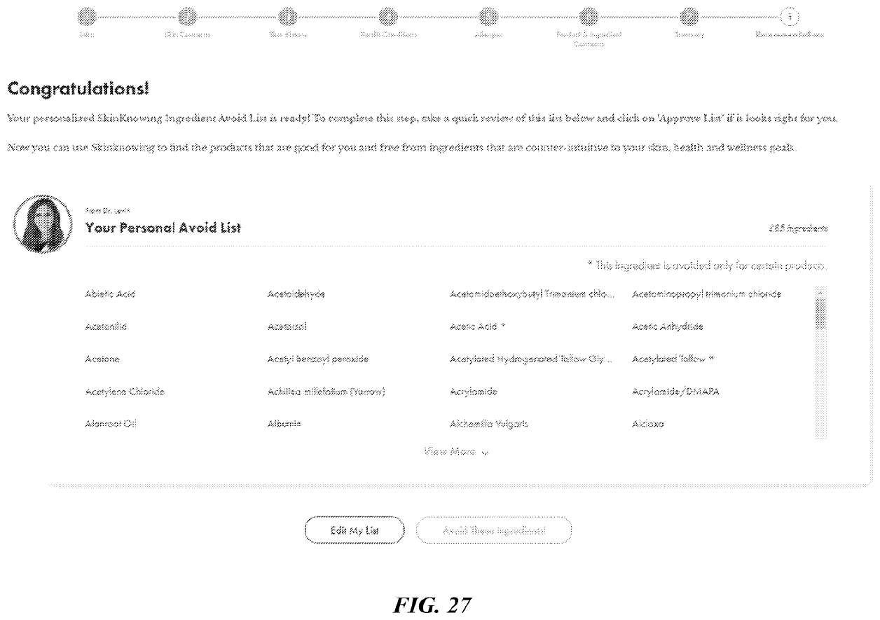

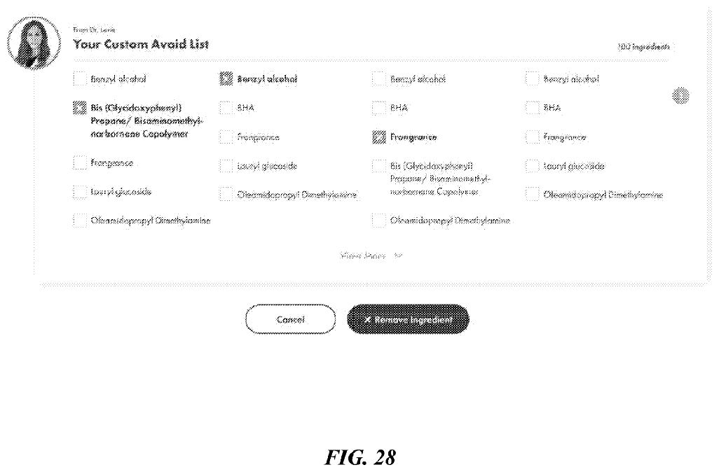

After receiving one or more inputs from the user, the UI may be configured to generate a custom ingredient avoid list for the user (FIG. 26). The UI may permit the user to edit the list and/or approve the custom ingredient avoid list (e.g., by selecting a button to affirmatively avoid the identified ingredients and any products containing those ingredients), as shown in FIG. 27 and FIG. 28. In one example, the UI may permit the user to remove one or more ingredients from the custom ingredient avoid list. In any of the embodiments described herein, the UI may permit the user to view, manage, and/or edit product recommendations. In any of the embodiments described herein, the UI may permit the user to view, manage, and/or edit personalized ingredient libraries. The UI disclosed herein may be configured to provide the user with a mapping of one or more user attributes (including individual attributes or groupings or combinations of attributes) to products or ingredients that are recommended as well as those that are not recommended.

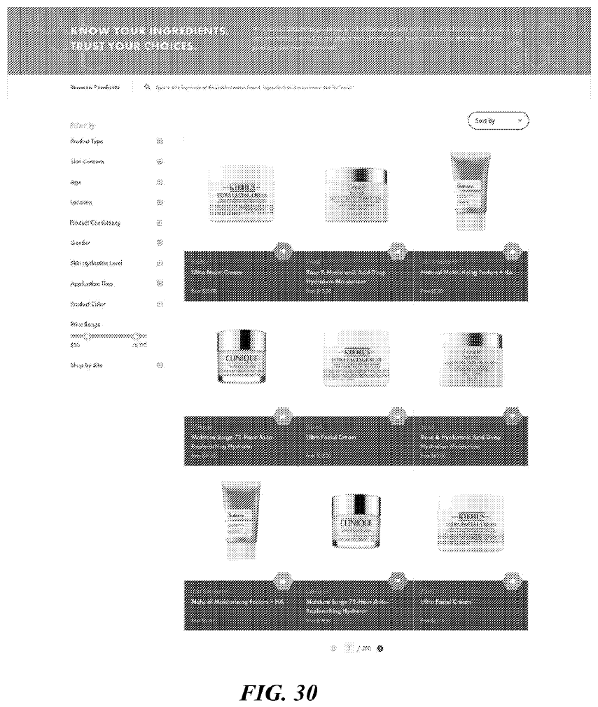

FIG. 30 illustrates an exemplary user interface for viewing and browsing products. A listing of products may be displayed to a user. The ingredients of the displayed products may be scanned and analyzed as described elsewhere herein to identify products that are okay to use, suggested, or therapeutic, as well as products that the user should avoid. Products that are okay to use, suggested, or therapeutic may be marked with a “thumbs up” icon, and products that the user should avoid may be marked with a “thumbs down” icon. The user interface may allow a user to browse through all of the products listed, search for products based on keywords (e.g., product name, brand, ingredients, skin concerns, etc.), sort search results, and/or filter through the products based on factors such as product type, skin concern, age, location, product consistency, gender, skin hydration level, application time, product color, and/or price range. The listing of products may include information on product name, a description of the product, brand name, and/or pricing information for each product.

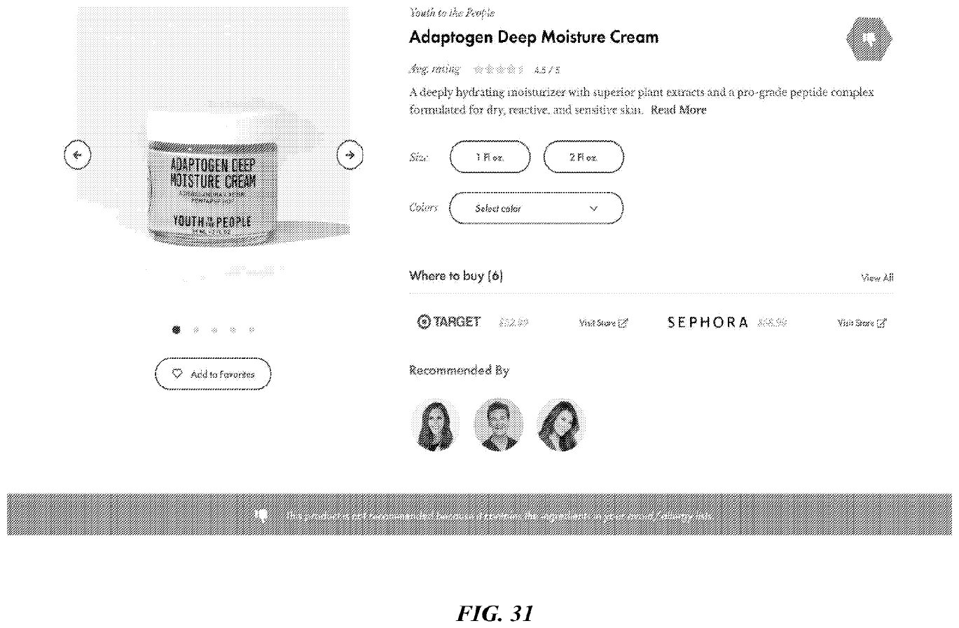

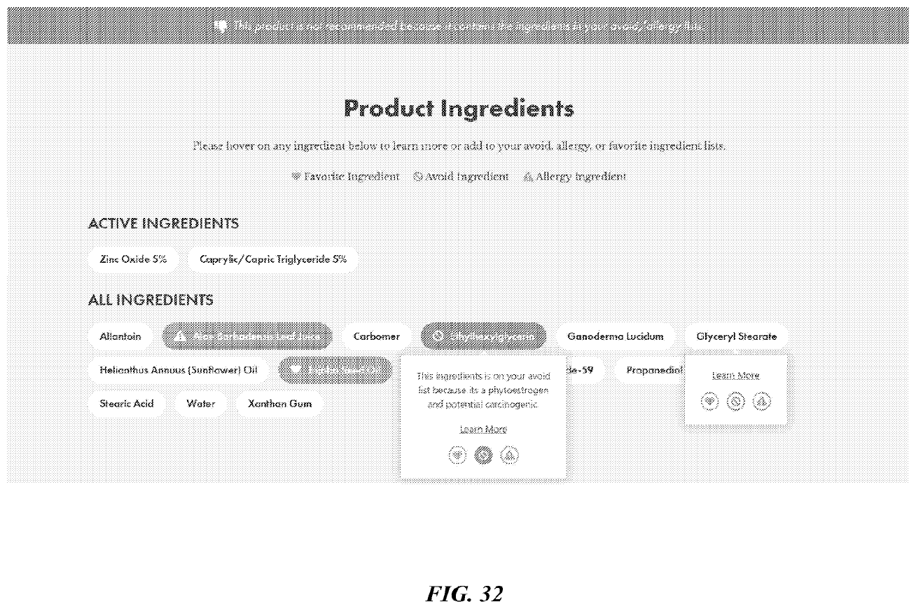

FIGS. 31-32 illustrate an exemplary user interface for viewing product and ingredient information. A user may select a product shown in the list of products generated by or displayed within the user interface, which may cause the user interface to display detailed information about the selected product and the ingredients of the product. The product information may include, for example, product name, brand name, product description, available sizes, available colors, a list of stores or retailers carrying stock of the product, and one or more qualified individuals who recommend the product for the user. The user interface may also allow a user to add the product to a favorites list. The user interface may also be configured to display information on whether the product is recommended or not recommended (e.g., because the product contains ingredients in a user's avoid list or allergy list).

In some cases, the user interface may display information about a product's ingredients. The user interface may display ingredient information, including a listing of active ingredients, a listing of all ingredients, and additional indications on which ingredients are the user's favorite ingredients, which ingredients are to be avoided by the user, and which ingredients the user is allergic to. The additional indications may comprise color-coding for the user's ease of review. When a user selects or hovers over a particular ingredient, the user interface may display additional information on why the ingredient is a suggested/therapeutic/okay to use ingredient, or why the ingredient is not a suggested/therapeutic/okay to use ingredient.

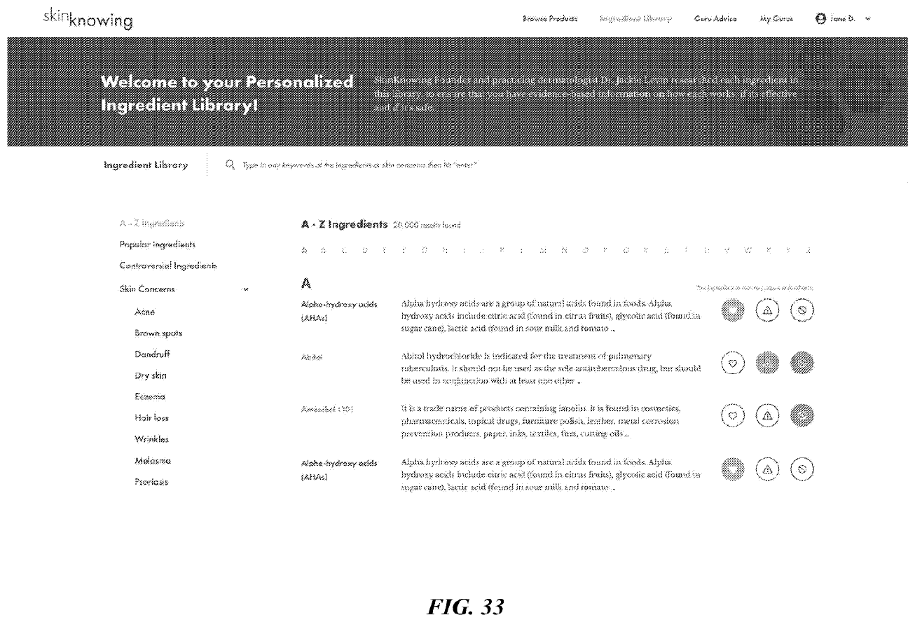

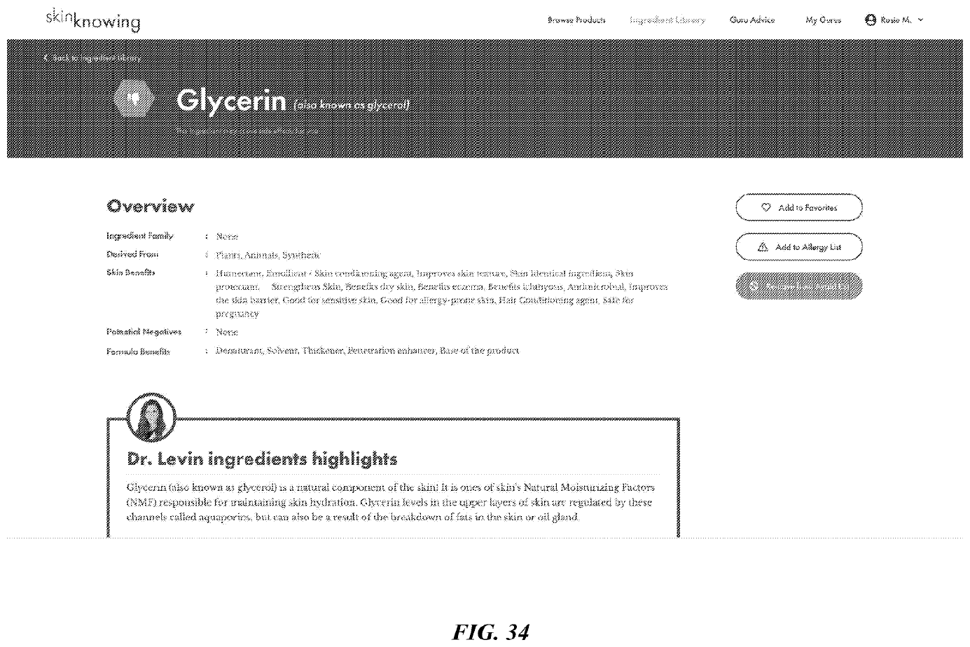

FIGS. 33-34 illustrate an exemplary user interface for viewing ingredient information for ingredients in an ingredient library. The user interface may be configured to display an ingredient library containing a list of all known ingredients for products (e.g., topical products, cosmetic products, or other clean beauty products). The ingredient list may include information on the ingredient name, a description of the ingredient, and an indication as to which ingredients are the user's favorite ingredients, which ingredients are to be avoided by the user, and which ingredients the user is allergic to. Such information may be color-coded for the user's ease of review. In some cases, the ingredient library may comprise a personalized ingredient library that is customized based on a user's attributes or preferences. In some cases, the ingredient library may be filtered so that the user can view a list of popular ingredients, controversial ingredients, and/or ingredients that can address certain health goals or concerns of the user (e.g., skin concerns such as acne, brown spots, dandruff, dry skin, eczema, hair loss, wrinkles, melasma, psoriasis, etc.).

In some cases, the user interface may be configured to display additional information about an ingredient if the user selects the ingredient. The user interface may provide information on the ingredient name, ingredient family, ingredient source (i.e., where the ingredient is derived from), skin benefits, potential negative effects, and/or formula benefits. The user interface may also indicate whether or not the ingredient is recommended for the user. In some cases, the user interface may also present one or more ingredient highlights that are generated or endorsed by a qualified individual. The user interface may permit the user to add the ingredient as a favorite, add the ingredient to an allergy list, or remove the ingredient from the user's avoid list.

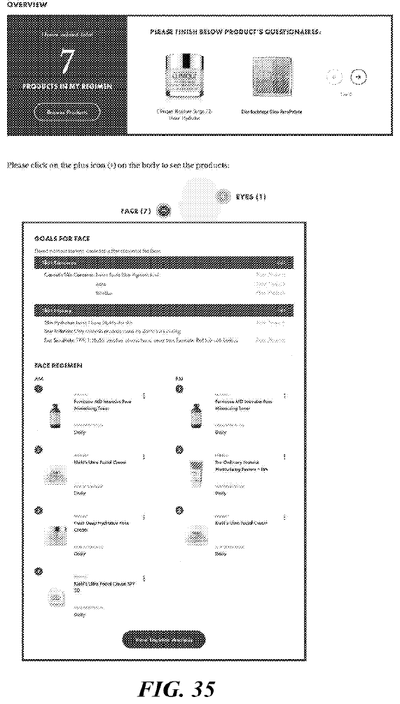

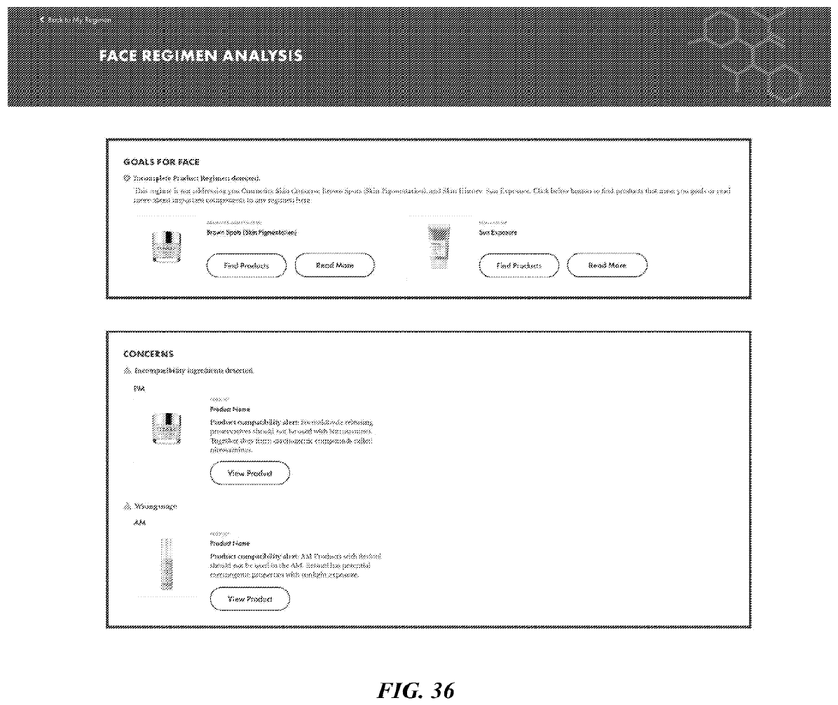

FIGS. 35-36 illustrate an exemplary user interface for viewing and managing a user's personalized regimen. The user interface may display a personalized regimen for a user based on the user's attributes and/or preferences. The personalized regimen may display a list of recommended products to use, the suggested frequency of use, and the timing of use (e.g., whether the product should be used in the morning, the afternoon, the evening, or any other time during the day or night). The user interface may also indicate various concerns identified for the user, display information on the health or medical history of the user, and present various products that are recommended for the user based on the identified concerns for the user and/or the user's health or medical history. In some cases, the user interface may provide an interactive visual representation of at least a portion of a user's body. The interactive visual representation may allow a user to click on different parts of the user's body to view the products that are suggested for those relevant locations on the user's body. In some cases, the user interface may prompt the user to answer additional questions about a product. Such product may or may not be added to or removed from the user's regimen, based on the additional information provided by the user.

In some cases, the user interface may present an analysis of a user's personalized regimen. The analysis may indicate, for instance, that an incomplete product regimen is detected. The incomplete product regimen may be due to the fact that certain products or ingredients do not address the user's concerns or goals. The user interface may allow a user to find other products that can help the user meet his or her goals or address any concerns. The user interface may also allow a user to read more about what ingredients or products would be helpful to improve the user's product regimen. In some cases, the user interface may also be configured to detect, for example, one or more incompatible ingredients (e.g., ingredients that are not compatible with the user's goals or concerns) and/or a wrong usage of an ingredient. The wrong usage may pertain to, for instance, a timing, a frequency, and/or a location of use or application.

Computer Systems

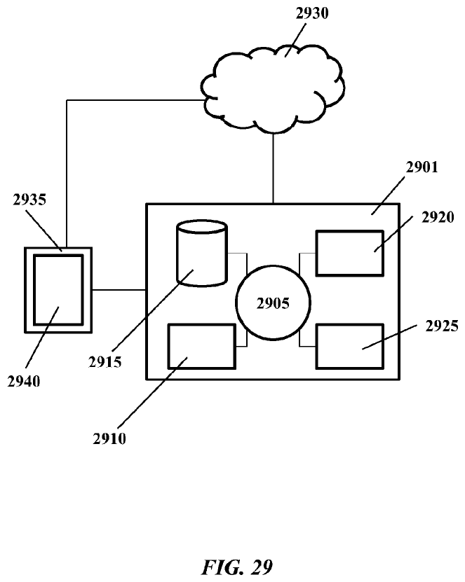

In an aspect, the present disclosure provides computer systems that are programmed or otherwise configured to implement methods of the disclosure, e.g., any of the subject methods for generating recommendations for ingredients and/or products. FIG. 29 shows a computer system 2901 that is programmed or otherwise configured to implement a method for generating recommendations for ingredients and/or products. The computer system 2901 may be configured to, for example, (a) receive information about a user, wherein the information comprises (i) genetic data of the user, (ii) user responses to a health and profile survey, and (iii) user inputs corresponding to one or more ingredients to avoid; (b) use a user analysis algorithm to generate one or more user attributes based on the information about the user; (c) correlate the one or more user attributes to one or more ingredient effects associated with one or more reference ingredients; and (d) use the correlations between the one or more user attributes and the one or more ingredient effects to generate (i) a preliminary ingredient avoid list. In some cases, the computer system may be further configured to generate a suggested ingredient avoid list by adding one or more cross reactors to the preliminary ingredient avoid list, wherein the one or more cross reactors comprise ingredients with a chemical structure similar to that of one or more ingredients in the preliminary ingredient avoid list. In some cases, the computer system may be further configured to generate a final ingredient avoid list by modifying the suggested ingredient avoid list based on one or more manual adjustments performed by the user. In some cases, the computer system may be further configured to generate a preliminary suggested ingredient list based on the correlations between the one or more user attributes and the one or more ingredient effects, wherein the preliminary suggested ingredient list comprises one or more ingredients with therapeutic effects. In some cases, the computer system may be further configured to generate an updated suggested ingredient list based on one or more user inputs corresponding to the user's favorite or preferred ingredients. In some cases, the computer system may be further configured to generate a final suggested ingredient list by subtracting the final ingredient avoid list from the updated suggested ingredient list. In some cases, the computer system may be further configured to compare (i) a list of ingredients associated with one or more products against (ii) the final ingredient avoid list and the final suggested ingredient list to generate a list or report of (iii) one or more recommended products for the user and (iv) one or more special conditions relevant to the user attributes. In some cases, the one or more recommended products may be identified based on whether certain ingredients are special condition avoid list ingredients and whether certain special conditions (e.g., for their method or location of application) are satisfied or not. The computer system 2901 can be an electronic device of a user or a computer system that is remotely located with respect to the electronic device. The electronic device can be a mobile electronic device.

The computer system 2901 may include a central processing unit (CPU, also “processor” and “computer processor” herein) 2905, which can be a single core or multi core processor, or a plurality of processors for parallel processing. The computer system 2901 also includes memory or memory location 2910 (e.g., random-access memory, read-only memory, flash memory), electronic storage unit 2915 (e.g., hard disk), communication interface 2920 (e.g., network adapter) for communicating with one or more other systems, and peripheral devices 2925, such as cache, other memory, data storage and/or electronic display adapters. The memory 2910, storage unit 2915, interface 2920 and peripheral devices 2925 are in communication with the CPU 2905 through a communication bus (solid lines), such as a motherboard. The storage unit 2915 can be a data storage unit (or data repository) for storing data. The computer system 2901 can be operatively coupled to a computer network (“net-work”) 2930 with the aid of the communication interface 2920. The network 2930 can be the Internet, an internet and/or extranet, or an intranet and/or extranet that is in communication with the Internet. The network 2930 in some cases is a telecommunication and/or data network. The network 2930 can include one or more computer servers, which can enable distributed computing, such as cloud computing. The network 2930, in some cases with the aid of the computer system 2901, can implement a peer-to-peer network, which may enable devices coupled to the computer system 2901 to behave as a client or a server.

The CPU 2905 can execute a sequence of machine-readable instructions, which can be embodied in a program or software. The instructions may be stored in a memory location, such as the memory 2910. The instructions can be directed to the CPU 2905, which can subsequently program or otherwise configure the CPU 2905 to implement methods of the present disclosure. Examples of operations performed by the CPU 2905 can include fetch, decode, execute, and writeback.

The CPU 2905 can be part of a circuit, such as an integrated circuit. One or more other components of the system 2901 can be included in the circuit. In some cases, the circuit is an application specific integrated circuit (ASIC).

The storage unit 2915 can store files, such as drivers, libraries and saved programs. The storage unit 2915 can store user data, e.g., user preferences and user programs. The computer system 2901 in some cases can include one or more additional data storage units that are located external to the computer system 2901 (e.g., on a remote server that is in communication with the computer system 2901 through an intranet or the Internet).

The computer system 2901 can communicate with one or more remote computer systems through the network 2930. For instance, the computer system 2901 can communicate with a remote computer system of a user (e.g., a consumer or potential consumer of healthcare, skincare, and/or cosmetic products). Examples of remote computer systems include personal computers (e.g., portable PC), slate or tablet PC's (e.g., Apple® iPad, Samsung® Gala29 Tab), telephones, Smart phones (e.g., Apple® iPhone, Android-enabled device, Blackberry®), or personal digital assistants. The user can access the computer system 2901 via the network 2930. In some cases, the computer system 2901 can communicate with a remote computer system that is located in or near a store kiosk, a cosmetics shop, a beauty products store, a doctor's office, or a dermatologist's office. In some cases, the computer system 2901 can further communicate with a remote database or server, which remote database or server may be configured to store one or more electronic medical records of a user.

Methods as described herein can be implemented by way of machine (e.g., computer processor) executable code stored on an electronic storage location of the computer system 2901, such as, for example, on the memory 2910 or electronic storage unit 2915. The machine executable or machine readable code can be provided in the form of software. During use, the code can be executed by the processor 2905. In some cases, the code can be retrieved from the storage unit 2915 and stored on the memory 2910 for ready access by the processor 2905. In some situations, the electronic storage unit 2915 can be precluded, and machine-executable instructions are stored on memory 2910.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 2901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2901 can include or be in communication with an electronic display 2935 that comprises a user interface (UI) 2940 for providing, for example, a portal for a user to provide one or more inputs usable to generate a user attribute, view one or more ingredient recommendations, view one or more product recommendations, review and modify one or more ingredient avoid lists, and/or review and modify one or more suggested ingredient lists. The portal may be provided through an application programming interface (API). A user or entity can also interact with various elements in the portal via the UI. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2905. The central processing unit 2905 may be located on a mobile device, a computer, or an imaging unit (e.g., a camera or a video camera). For example, the algorithm may be configured to implement a method for generating recommendations for ingredients or products. The recommendations may be provided in a report that can be sent to a computing device or a mobile device. The method may comprise (a) receiving information about a user, wherein the information comprises (i) genetic data of the user, (ii) user responses to a health and profile survey, and (iii) user inputs corresponding to one or more ingredients to avoid; (b) using a user analysis algorithm to generate one or more user attributes based on the information about the user; (c) correlating the one or more user attributes to one or more ingredient attributes associated with one or more reference ingredients; and (d) using the correlations between the one or more user attributes and the one or more ingredient attributes to generate (i) a preliminary ingredient avoid list. In some cases, the method may further comprise generating a suggested ingredient avoid list by adding one or more cross reactors to the preliminary ingredient avoid list, wherein the one or more cross reactors comprise ingredients with a chemical structure similar to that of one or more ingredients in the preliminary ingredient avoid list. In some cases, the method may further comprise generating a final ingredient avoid list by modifying the suggested ingredient avoid list based on one or more manual adjustments performed by the user. In some cases, the method may further comprise generating a preliminary suggested ingredient list based on the correlations between the one or more user attributes and the one or more ingredient effects, wherein the preliminary suggested ingredient list comprises one or more ingredients with therapeutic effects. In some cases, the method may further comprise generating an updated suggested ingredient list based on one or more user inputs corresponding to the user's favorite or preferred ingredients. In some cases, the method may further comprise generating a final suggested ingredient list by subtracting the final ingredient avoid list from the updated suggested ingredient list. In some cases, the method may further comprise comparing (i) a list of ingredients associated with one or more products against (ii) the final ingredient avoid list and the final suggested ingredient list to generate (iii) one or more product recommendations.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1: A computerized method of personalized skincare management for an individual user, the method comprising:
obtaining, in a host processor from a user device, selected personal information from the user;
determining, via the host processor, an individualized set of user attributes for the user based at least in part on (i) the user's personal information and (ii) an analysis or interpretation of one or more combinations or groupings of user inputs or user attributes derived from the user's personal information;
assigning, via the host processor, one or more favorable correlations and one or more unfavorable correlations between (i) ingredients used in topical products and/or one or more effects or properties of the ingredients and (ii) one or more of the user attributes;
determining, via the host processor, a personalized preliminary avoid list of ingredients that the user should avoid when purchasing skincare products by referencing the assigned user attributes to the unfavorable correlations;
determining, via the host processor, at least one cross reactor to at least one ingredient included within the personalized preliminary ingredient avoid list, wherein the at least one cross reactor includes an ingredient with a chemical structure similar to the at least one ingredient included within the personalized preliminary ingredient avoid list;
determining, via the host processor, an ingredient to conditionally avoid from the at least one ingredient included within the personalized preliminary ingredient avoid list, the ingredient to conditionally avoid corresponding to an ingredient that meets a specific condition related to a product attribute of a skincare product,
creating, via the host processor, a personalized avoid list of ingredients by adding the determined at least one cross reactor to the personalized preliminary ingredient avoid list and labeling the ingredient to conditionally avoid in addition to the related product attribute;
generating, via the host processor, a personalized recommended or okay to use list of ingredients that the user should consider when purchasing skincare products by referencing the assigned user attributes to the favorable correlations; and
transmitting, from the host processor to the user device, the personalized avoid list and the personalized recommended or okay to use list.

2: The method of claim 1, further comprising:
indexing a skincare products clearinghouse computer database according to the user avoid list; and
defining a user avoid list of skincare products from the indexing the skincare products clearinghouse computer database step.

3-4. (canceled)

5: A method for generating recommendations for ingredients or products, the method comprising:
(a) receiving, in a host processor from a user device, information about a user, wherein the information comprises (i) genetic data of the user, (ii) allergy information, (iii) user medical history, current medical diagnoses, prescriptions, and/or other information or data received from a data source or an application programming interface of an electronic record system, (iv) user responses to a health and profile survey, and (v) user inputs corresponding to one or more ingredients to avoid;
(b) using, via the host processor, a user analysis algorithm to generate one or more user attributes based on (i) the information about the user and/or (ii) one or more inferences or other user attributes derivable from the information about the user;
(c) correlating, via the host processor, the one or more user attributes to one or more ingredient effects associated with one or more reference ingredients;
(d) using, via the host processor, the correlations between the one or more user attributes and the one or more ingredient effects to generate a preliminary ingredient avoid list;
(e) generating, via the host processor, an ingredient avoid list by adding one or more cross reactors to the preliminary ingredient avoid list, wherein the one or more cross reactors comprise ingredients with a chemical structure similar to that of one or more ingredients in the preliminary ingredient avoid list; and
(g) determining, via the host processor, an ingredient to conditionally avoid from the ingredient avoid list, the ingredient to conditionally avoid corresponding to an ingredient that meets a specific condition related to a product attribute.

6. (canceled)

7: The method of claim 5, further comprising generating a final ingredient avoid list by modifying the ingredient avoid list based on one or more manual adjustments performed by the user.

8: The method of claim 7, further comprising generating a preliminary suggested ingredient list based on the correlations between the one or more user attributes and the one or more ingredient effects, wherein the preliminary suggested ingredient list comprises one or more ingredients with therapeutic effects.

9: The method of claim 8, further comprising generating an updated suggested ingredient list based on one or more user inputs corresponding to the user's favorite or preferred ingredients.

10: The method of claim 9, further comprising generating a final suggested ingredient list by subtracting the final ingredient avoid list from the updated suggested ingredient list.

11: The method of claim 10, further comprising comparing (i) a list of ingredients associated with one or more products against (ii) the final ingredient avoid list and the final suggested ingredient list to generate (iii) one or more product recommendations.

12: The method of claim 11, wherein the one or more product recommendations comprise an indication that a product is not a recommended product.

13: The method of claim 11, wherein the one or more product recommendations comprise an indication that a product is a recommended product or an okay to use product.

14: The method of claim 11, wherein the one or more product recommendations comprise an indication that a product is a suggested product or a potential therapeutic product.

15: The method of claim 11, wherein the list of ingredients associated with one or more products is compiled by merging product data or ingredient data from a plurality of sources.

16: The method of claim 11, wherein the comparison comprises determining whether a product has any ingredients listed in the final ingredient avoid list.

17: The method of claim 11, wherein the comparison comprises determining whether a product has any ingredients listed in the final suggested ingredient list.

18: The method of claim 11, wherein the one or more product recommendations are generated based on one or more product attributes.

19: The method of claim 18, wherein the one or more product attributes comprise a form of application and a location of application.

20: The method of claim 8, wherein the correlations between the one or more user attributes and the one or more ingredient effects are derived in part by (i) interpreting one or more mechanisms involved or associated with the one or more user attributes and (ii) determining which ingredients affect the one or more mechanisms positively or negatively.

21: The method of claim 1, wherein the favorable correlations and the unfavorable correlations are determined based at least in part on one or more attributes of the topical products.

22: The method of claim 21, wherein the one or more attributes correspond to a method of using or applying the topical products or a location of application or use for the topical products.

23: The method of claim 1, further comprising generating one or more alerts for incompatibility amongst ingredients in a product formulation and for chemical reactions involving ingredients that are negatives for the user, based at least in part on the individualized set of user attributes for the user.

24: The method of claim 23, wherein the individualized set of user attributes correspond to or relate to at least one of wellness, allergies, moral concerns, or health goals.

25: The method of claim 1, further comprising generating one or more ingredient and product recommendations based on one or more special conditions, wherein the one or more special conditions relate to (i) a location in which the ingredients or products are used or applied, (ii) other ingredients in the products, or (iii) ingredients in other products used in a same location and/or a same time of day.

26: The method of claim 1, further comprising interpreting a level of risk for the user for one or more health conditions or diseases based on user genetics or heath history factors that affect a threshold of evidence standard for ingredients the user should avoid.

27-29. (canceled)
